(19) United States
(12) Patent Application Publication
(10) Pub. No.: US 2026/0139317 A1
(43) Pub. Date: May 21, 2026

(54) Method of Determining Loss of Heterozygosity Status of a Tumor

(71) Applicants: VIB VZW, Gent (BE); Katholieke Universiteit Leuven, Leuven (BE)

(72) Inventors: Diether Lambrechts, Leuven (BE); Tom Venken, Leuven (BE); Pieter Busschaert, Leuven (BE); Liselore Loverix, Leuven (BE); Toon Van Gorp, Leuven (BE); Ignace Vergote, Leuven (BE)

(21) Appl. No.: 19/122,416

(22) PCT Filed: Oct. 19, 2023
(86) PCT No.: PCT/EP2023/079121

§ 371 Date: Apr. 18, 2025

(30) Foreign Application Priority Data
Oct. 19, 2022 (EP) 22202464.8

(51) Int. Cl. C12Q 1/6886; G16B 40/20

(52) U.S. Cl. CPC C12Q 1/6886; G16B 40/20; C12Q 2600/156

(57) ABSTRACT

This disclosure relates to methods of determining the loss of heterozygosity (LOH) status of a tumor and to methods of determining the homologous recombination repair deficiency (HRD) status of a tumor. Such methods further translate into ways of predicting the response of a subject having cancer to treatments or therapies comprising DNA damaging agents and/or agents inhibiting or impairing DNA repair; and into use of such treatments or therapies when the LOH or HRD status of the subject having cancer is positive.

### CROSS REFERENCE TO RELATED APPLICATIONS

This application is a national phase entry under 35 U.S.C. § 371 of International Patent Application PCT/EP2023/079121, filed Oct. 19, 2023, designating the United States of America and published in English as International Patent Publication WO2024/083971 on Apr. 25, 2024, which claims the benefit under Article 8 of the Patent Cooperation Treaty to European Patent Application Serial No. 22202464.8, filed Oct. 19, 2022, the entireties of which are hereby incorporated by reference.

### FIELD OF THE INVENTION

This disclosure relates to methods of determining the loss of heterozygosity (LOH) status of a tumor and to methods of determining the homologous recombination repair deficiency (HRD) status of a tumor. Such methods further translate into ways of predicting the response of a subject having cancer to treatments or therapies comprising DNA damaging agents and/or agents inhibiting or impairing DNA repair; and into use of such treatments or therapies when the LOH or HRD status of the subject having cancer is positive.

### BACKGROUND OF THE INVENTION

It is generally known that different patients having the same type of cancer respond differently to the same therapy. This is true for chemotherapy as well as for the more recent immunotherapies including immune checkpoint inhibitors. This led cancer researchers worldwide to identification of biochemical or genetic markers that can predict response to a particular anti-cancer therapy or that can identify whether a patient on a particular anti-cancer therapy is responding to that therapy, preferably early after initiation of that therapy.

Such successful prediction or early identification of response to therapy, when available, not only improves the outcome for the patients, but also results in health-economic benefits.

One field of genetic testing in the context of predicting therapy response relates to testing of genomic scarring or homologous recombination (repair) deficiency (HRD; homologous recombination repair (HRR) is the process of normal DNA repair, especially of repair of breaks in both DNA strands) testing in the context of predicting response to therapies including poly-ADP-ribose polymerase inhibitors (PARPi), platinum-based therapies, etc.

A number of HRD causes have been identified including mutations or lowered expression of the BRCA1 and/or BRCA2 genes and other genes such as RAD51C and PALB2. HRD is, however, not always linked to identifiable gene mutations. Across cancers, HRD occurs at a frequency of about 6%. Rates can be as high as 30% for ovarian cancer, and intermediate for breast, pancreatic and prostate cancer (12-13%).

A number of genomic scarring parameters have been introduced including loss of heterozygosity (LOH) or percentage LOH (see e.g. WO2011/106541; WO2011/160063; WO2013/096843; Abkevich et al. 2012, Br J Cancer 107:1776-1782), fractional LOH (FLOH; e.g. Wang et al. 2012, Clin Cancer Res 18:5806-5815), telomeric allelic imbalance (TAI; see e.g. WO 2012/027224; WO2013/130347; Birkbak et al. 2012, Cancer Discov 2:366-375), large scale transitions (LST; see e.g. WO2013/182645; WO 2015/086473; Popova et al. 2012, Cancer Res 72:5454-5462), and combinations thereof (see e.g. WO2014/165785; WO2016/025958 and US 20170283879; Timms et al. 2014, Breast Cancer Res 16:475). These documents linked diagnostic test outcome to prediction of outcome of treatment outcome, in particular to outcome of treatment with platinum-based therapeutic modalities.

Few commercial assays testing a patient's HRD status are available and include the Myriad Genetics MyChoice® CDx test (considered as the gold standard in the field; based on LOH, TAI, LST, and status of BRCA1 and BRCA2) and the FoundationOne® CDx test (based on copy number alterations in a set of genes (including HRR genes) and % LOH). According to c n y c (version 2 Aug. 2022), however, methods based on % LOH determination are significantly less successful in determining HRD than the Myriad Genetics MyChoice® CDx test, which is confirmed in Stover et al. 2020 (Gynecologic Oncology 159:887-898). Further methodologies for determining LOH status of a DNA sample involve deterministic restriction-site whole genome amplification as disclosed in WO2021/019459.

A comprehensive overview of clinical assays applied in determining HRD status is provided in Stover et al. 2020 (Gynecologic Oncology 159:887-898).

### SUMMARY OF THE INVENTION

The invention relates to methods for determining the loss-of-heterozygosity (LOH) status of a genomic DNA sample, such methods comprising the steps of:
dividing the sequence of the genomic DNA in a plurality of bins of the same size;
determining the LOH status of each bin, therewith providing a set of LOH/bin features;
computer-implemented classification of the (genomic DNA based on its) set of LOH/bin features with a data classification algorithm, wherein the data classification algorithm is trained on a set of reference genomic DNA samples including genomic DNA samples known of being LOH positive and/or homologous recombination repair deficient (HRD positive) and including genomic DNA samples known of being LOH negative and/or homologous recombination repair proficient (HRD negative);
determining the genomic DNA sample to be LOH positive or to have a LOH positive status when the data classification algorithm classifies the genomic DNA sample as most closely resembling reference genomic DNA known of being LOH positive and/or HRD positive; or determining the genomic DNA sample to be LOH negative or to have a LOH negative status when the data classification model classifies the genomic DNA sample as most closely resembling reference genomic DNA known of being LOH negative and/or HRD negative.

In one embodiment thereto, the size of the bins is equal to or smaller than 1.5 Mb.

Alternative methods for determining the loss-of-heterozygosity (LOH) status of a genomic DNA sample, are methods comprising the steps of:
dividing the sequence of the genomic DNA in a plurality of bins of the same size;
determining the LOH status of each bin, therewith providing a set of LOH/bin features;
classification of the set of LOH/bin features relative to a set of reference genomic DNA samples including genomic DNA samples known of being LOH positive and/or HRD positive and including genomic DNA samples known of being LOH negative and/or HRD negative;
determining the genomic DNA sample to be LOH positive or to have a LOH positive status when the classification classifies the genomic DNA sample as most closely resembling reference genomic DNA known of being LOH positive and/or HRD positive; or determining the genomic DNA sample to be LOH negative or to have a LOH negative status when the classification classifies the genomic DNA sample as most closely resembling reference genomic DNA known of being LOH negative and/or HRD negative;
   wherein the size of bins is equal to or smaller than 1.5 Mb.

In these methods, the LOH status of a bin is in one embodiment based on the copy number of one or more single nucleotide polymorphisms (SNPs) of interest, and is determined as the mean of the LOH positive SNPs relative to all SNPs of interest within the bin, wherein a SNP is LOH positive or has LOH positive status when the copy number of the minor allele of the SNP is zero. Herein, the copy number of a SNP of interest can for instance be derived from the copy number of a segment of the genomic DNA with constant copy number comprising the SNP of interest. Herein, SNPs can for example be captured by hybridization to a plurality of oligonucleotide probes.

In these methods, the LOH status of a bin is in another embodiment based on the copy number of a nucleotide position in the bin, and is determined as the copy number of a segment of the genomic DNA with constant copy number comprising the nucleotide position in the bin, wherein a bin is LOH positive or has LOH positive status when the copy number of the minor allele of the nucleotide position in the bin is zero.

In these methods, the copy number (of a SNP, of a nucleotide position in the bin) can be determined based on untargeted or targeted sequencing of the genomic DNA.

The above methods in particular are methods for determining the homologous recombination deficiency (HRD) status of the genomic DNA sample, wherein a genomic DNA sample classified as being LOH positive or as having a LOH positive status is HRD positive or is having a HRD positive status.

The above methods can furthermore be included in or be part of methods of predicting the response of a subject or patient having cancer or a tumor to a treatment or treatment regimen comprising a DNA damaging agent and/or an agent inhibiting or impairing DNA repair, wherein the subject or patient is likely to respond to the treatment or treatment regimen when a genomic DNA sample obtained from the tumor or from a cancer cell of the cancer or tumor is determined to have a LOH positive status, or to have a HRD positive status as outlined hereinabove.

The above methods can furthermore be included in or be part of methods of determining or assessing survival probability of a subject or patient having cancer or a tumor receiving treatment or a treatment regimen comprising a DNA damaging agent or therapy and/or comprising an agent inhibiting or impairing DNA repair or therapy comprising inhibiting or impairing DNA repair, wherein the subject or patient (having the cancer or tumor) is likely to have an increased survival probability if a genomic DNA sample obtained from the tumor or from a cancer cell of the cancer or tumor is determined to have a LOH positive status, or to have a HRD positive status as outlined hereinabove.

Any of the above methods can be extended to further comprise determining the LOH status of each chromosome, therewith providing a set of LOH/chromosome features; and including the set of LOH/chromosome features in the classification.

Any of the above methods can be extended to further comprise determining the LOH status of the genomic DNA, therewith providing a LOH/genome feature; and including the LOH/genome feature in the classification.

The invention further relates to DNA damaging agents and/or an agents inhibiting or impairing DNA repair for use in treating a tumor or cancer if a genomic DNA sample obtained from the tumor or from a cancer cell of the cancer or tumor is determined to have a LOH positive status, or to have a HRD positive status as outlined hereinabove.

The invention further relates to computer programs having instructions which when executed cause a computing or data processing system or device to perform any of the above methods, or to perform a step of any of the above methods.

The invention further relates to computing or data processing systems or devices, machine readable media, or diagnostic kits comprising a means for carrying out or performing any of the above methods, or for carrying out or performing a step of any of the above methods.

The invention further relates to computer systems, machine readable media, or diagnostic kits comprising a data classification algorithm trained to classify a genomic DNA sample as being LOH or HRD positive or as being LOH or HRD negative, wherein the data classification algorithm was trained based on the input features comprising a set of LOH/bin features, and optionally sets of LOH/chromosome features and/or sets of LOH/genome features, of a set of reference genomic DNA samples including genomic DNA samples known of being LOH positive and/or HRD positive and including genomic DNA samples known of being LOH negative and/or HRD negative.

### BRIEF DESCRIPTION OF THE DRAWINGS

FIGS. 1A and 1B. Distinguishing HRD-positive and HRD-negative samples by means of (FIG. 1A) fractional loss-of-heterozygosity (FLOH, calculated by the ASCAT algorithm), and by means of (FIG. 1B) the scarHRD-score, with indication of AUC and P-values. Analysis was performed on 192 samples of the PAOLA-1 trial for which the Myriad myChoice HRD status (“positive” or “negative”) was known.

FIGS. 2A and 2B. ROC curves of Myriad myChoice positive versus Myriad myChoice negative samples. ROC curves obtained by means of LOH as determined by number of analyzing LOH regions of intermediate size, also known as “HRD-LOH” (FIG. 2A, “median AUC”), by means of analyzing LOH regions of intermediate size and large-scale transition (LST) regions (FIG. 2B, “median AUC”), and by means of analyzing LOH regions of intermediate size, large-scale transition (LST) regions and telomeric allelic imbalance (TAI) regions (FIG. 2C, “median AUC”). In all cases, this was compared with the LOH+LST+TAI analysis via the scarHRD algorithm (“AUC original score”). Analysis was performed on 192 samples of the PAOLA-1 trial (same as for FIG. 1) for which the Myriad myChoice HRD status (“positive” or “negative”) was known. All combined “median” scores were generated using logistic regression and 10-fold cross-validation.

FIG. 3. Flowchart of the exemplary sequencing and analysis pipeline. In total, 192 FFPE samples from the PAOLA1 trial with known Myriad myChoice status were processed. GATK: Genome Analysis Toolkit; dedup: deduplicate.

FIG. 4. ROC curves of Myriad myChoice positive versus Myriad myChoice negative samples. HRD score: as determined with scarHRD algorithm; PAM (Partitioning Around Medoids algorithm) score: classification based on mean LOH/bin, on mean LOH/chromosome and on mean genome-wide LOH features. Set of 82 Myriad myChoice HRD-negative and 110 Myriad myChoice HRD-positive samples.

FIGS. 5A and 5B. Survival curve analysis of olaparib arm (FIG. 5A) and placebo arm (FIG. 5B). Survival probabilities were calculated with respect to classification using the Myriad myChoice HRD status or the PAM derived score (see FIG. 4) based on mean LOH/bin, on mean LOH/chromosome and on mean genome-wide LOH features. HRD+: homologous recombination deficient or HRD positive; HRD−: homologous recombination proficient or HRD negative.

FIGS. 6A and 6B. Survival curve analysis of olaparib arm (FIG. 6A) and placebo arm (FIG. 6B). Survival probabilities were calculated with respect to classification using the Myriad myChoice HRD status or the PAM derived score mean LOH/bin features only. HRD+: homologous recombination deficient or HRD positive; HRD−: homologous recombination proficient or HRD negative. Bin-size: 1 Mb (1000 kbp).

FIG. 7. Distinguishing HRD-positive and HRD-negative samples by means of Random Forest (RF) based classification of the LOH/bin features. Analysis was performed on 192 samples of the PAOLA-1 trial for which the Myriad myChoice HRD status (“positive” or “negative”) was known.

FIGS. 8A and 8B. Survival curve analysis of olaparib arm (FIG. 8A) and placebo arm (FIG. 8B). Survival probabilities were calculated with respect to classification using the Myriad myChoice HRD status or the Random Forest based classification of the LOH/bin features. HRD+: homologous recombination deficient or HRD positive; HRD−: homologous recombination proficient or HRD negative; PFS: progression free survival.

FIGS. 9A-9D. Survival curve analysis of olaparib arm (FIG. 9A, FIG. 9C) and placebo arm (FIG. 9B, FIG. 9D) As in FIG. 8. (FIG. 8A) and (FIG. 8B) bin-size: 0.75 Mb (750 kbp). (FIG. 9C) and (FIG. 9D) bin-size: 1.25 Mb (1250 kbp).

FIGS. 10A-10F. Survival curve analysis of olaparib arm (FIG. 10A, FIG. 10C, FIG. 10E) and placebo arm (FIG. 10B, FIG. 10D, FIG. 10F) As in FIG. 6. (FIG. 6A) and (FIG. 6B) bin-size: 0.5 Mb (500 kbp). (FIG. 10C) and (FIG. 10D) bin-size: 0.75 Mb (750 kbp). (FIG. 10E) and (FIG. 10F) bin-size: 1.25 Mb (1250 kbp).

### DETAILED DESCRIPTION

Existing homologous recombination (repair) deficiency (HRD) tests assess either the cause (e.g. screening homologous recombination repair (HRR) gene panel for presence of mutations), the result of HRD (i.e., genomic scarring), or both. Whereas HRD testing has been linked with predicting outcome of some cancer therapies, such predictive results are still not optimal and in general, mutations in most HRR genes, although informative about a patient's general status, have not yet solidly been assigned such predictive power (Miller et al. 2020, Annals Oncol 31:1606-1622). Most assays to identify HRD are relying on multiple different features (e.g. LOH, LST and TAI), and those including a % LOH feature (e.g. WO2011106541 refers to determination of % LOH and a sample was defined as having high LOH when >35% of the genome in cells of the sample has LOH) were reported to underperform (see Introductory section hereinabove).

The objective of the work leading to the invention was to design a relatively simple assay to determine the LOH status of the DNA in a tumor as a proxy for determining the HRD status of that tumor. The further objective, one perceived as difficult if not impossible to reach in view of all previous work done in the field, was that such assay should nevertheless perform at least equally well in determining HRD status as the current gold standard assay implying LOH, LST, and TAI features. In particular, prior methods for determining LOH-status of the DNA in a tumor sample (i) only included regions of LOH of a size larger than 1.5 Mb (as shorter regions of LOH were reported not to correlate with HR-deficiency) and more in particular LOH-regions having a size of larger than 1.5 Mb and shorter than a full chromosome (e.g. WO2011160063A2, WO2013096843A1), although in practice the low-end size threshold more likely is 15 Mb (Abkevich et al. 2012, Br J Cancer 107:1776-1782); or (ii) included calculation of fractional or percentage LOH in the DNA of a tumor sample reported to underperform (e.g. Stover et al. 2020, Gynaecologic Oncology 159:887-898). It was surprisingly found that a trained machine learning algorithm could reliably determine LOH or HRD status of the DNA of a tumor based on the LOH status of genomic DNA bins of equal size of 1.5 Mb. Two different machine learning algorithms led to equally reliable LOH or HRD status determination, both further equally reliable as the current gold standard assay implying LOH, LST, and TAI features. Moreover further surprising, the bin size could be reduced to smaller than 1.5 Mb(p), as equal results were obtained with exemplary bin sizes of 1.25 Mb(p) (1250 kb(p)), 1 Mb(p) (1000 kb(p)), 0.75 Mb(p) (750 kb(p)) and 0.5 Mb(p) (500 kb(p)).

In a first aspect, the invention relates to methods for determining the loss-of-heterozygosity (LOH) status of a (test) genomic DNA sample, comprising the steps of:
dividing the sequence of the (test) genomic DNA in a plurality of bins of the same size or of equal size;
determining the LOH status of each bin, therewith providing a set of LOH/bin features;
computer-implemented data classification model/algorithm analysis of the set of LOH/bin features,
   wherein the data classification model/algorithm is or was trained on a set of reference genomic DNA samples including genomic DNA samples known of being LOH positive and/or of being homologous recombination repair deficient (HRD positive) and including genomic DNA samples known of being LOH negative and/or of being homologous recombination repair proficient (HRD negative), or
   wherein the data classification model/algorithm is or was trained based on the input features comprising the (a set of) LOH/bin features of a set of reference genomic DNA samples including genomic DNA samples known of being LOH positive and/or HRD positive and including genomic DNA samples known of being LOH negative and/or HRD negative;
   or, alternatively:
   computer-implemented classification of the set of LOH/bin features (or more precisely of the test genomic DNA based on its set of LOH/bin features) with a data classification model/algorithm, wherein the data classification model/algorithm is or was trained on a set of reference genomic DNA samples (or more precisely is or was trained for classifying (a set of) reference genomic DNA samples as being LOH or HRD positive, or as LOH or HRD negative), wherein the reference genomic DNA samples are including genomic DNA samples known of being LOH positive and/or HRD positive and including genomic DNA samples known of being LOH negative and/or HRD negative, or
   wherein the data classification model/algorithm is or was trained based on the input features comprising the (a set of) LOH/bin features of a set of reference genomic DNA samples including genomic DNA samples known of being LOH positive and/or HRD positive and including genomic DNA samples known of being LOH negative and/or HRD negative;
   or, alternatively:
computer-implemented classification, inferencing or scoring of the set of LOH/bin features of the (test) genomic DNA (or more precisely, classification, inferencing or scoring of the (test) genomic DNA base on its set of LOH/bin features) with a trained data classification algorithm or model, wherein the trained data classification algorithm or model is or was trained for classifying (as LOH or HRD positive, or as LOH or HRD negative) (a set of) reference genomic DNA samples including genomic DNA samples known of being LOH positive and/or HRD positive and including genomic DNA samples known of being LOH negative and/or HRD negative;
determining the (test) genomic DNA sample to be LOH positive or to have a LOH positive status when the (trained) data classification model/algorithm (or when the data classification of the above or previous step) classified, inferenced or scored the (test) genomic DNA sample as most closely resembling reference genomic DNA known of being LOH positive and/or HRD positive; or determining the (test) genomic DNA sample to be LOH negative or to have a LOH negative status when the (trained) data classification model/algorithm (or when the data classification of the above or previous step) classifies, inferenced or scored the (test) genomic DNA sample as most closely resembling reference genomic DNA known of being LOH negative and/or HRD negative.

These methods can be alternatively defined as methods for determining the loss-of-heterozygosity (LOH) status of a (test) genomic DNA sample, comprising the steps of:
dividing the sequence of the (test) genomic DNA in a plurality of bins of the same size or of equal size;
determining the LOH status of each bin, therewith providing a set of LOH/bin features;
computer-implemented data classification model/algorithm analysis of the set of LOH/bin features (or more precisely of the test genomic DNA based on its set of LOH/bin features); or, alternatively, computer-implemented classification, inferencing or scoring of the set of LOH/bin features (or more precisely of the test genomic DNA based on its set of LOH/bin features) with a (trained) data classification model/algorithm;
determining the (test) genomic DNA sample to be LOH positive or to have a LOH positive status when the (trained) data classification model/algorithm (or when the data classification of the above or previous step) classified, inferences or scored the (test) genomic DNA sample as most closely resembling reference genomic DNA known of being LOH positive and/or HRD positive; or determining the (test) genomic DNA sample to be LOH negative or to have a LOH negative status when the (trained) data classification model/algorithm (or when the data classification of the above or previous step) classified, inferenced or scored the (test) genomic DNA sample as most closely resembling reference genomic DNA known of being LOH negative and/or HRD negative;
   wherein the data classification model/algorithm is or was trained on a set of reference genomic DNA samples (or more precisely is or was trained for classifying (as LOH or HRD positive, or as LOH or HRD negative) (a set of) reference genomic DNA samples), including genomic DNA samples known of being LOH positive and/or HRD positive and including genomic DNA samples known of being LOH negative and/or HRD negative, or
   wherein the data classification model/algorithm is or was trained based on the input features comprising the (a set of) LOH/bin features of a set of reference genomic DNA samples including genomic DNA samples known of being LOH positive and/or HRD positive and including genomic DNA samples known of being LOH negative and/or HRD negative.

In one embodiment to these methods, the size of the bins is smaller than 1.5 Mb.

An alternative first aspect relates to methods for determining the loss-of-heterozygosity (LOH) status of a (test) genomic DNA sample, such methods comprising the steps of:
dividing the sequence of the (test) genomic DNA in a plurality of bins of the same size;
determining the LOH status of each bin, therewith providing a set of LOH/bin features;
classification of the set of mean LOH/bin features relative to a set of reference genomic DNA samples including genomic DNA samples known of being LOH positive and/or HRD positive and including genomic DNA samples known of being LOH negative and/or HRD negative; or classification of the (test) genomic DNA based on its set of LOH/bin features, relative to a set of reference genomic DNA samples including genomic DNA samples known of being LOH positive and/or HRD positive and including genomic DNA samples known of being LOH negative and/or HRD negative;
determining the (test) genomic DNA sample to be LOH positive or to have a LOH positive status when the classification (or when the classification of the above or previous step) classified the (test) genomic DNA sample as most closely resembling reference genomic DNA known of being LOH positive and/or HRD positive; or determining the (test) genomic DNA sample to be LOH negative or to have a LOH negative status when the classification (or when the classification of the above or previous step) classified the (test) genomic DNA sample as most closely resembling reference genomic DNA known of being LOH negative and/or HRD negative;
   wherein the size of the bins is smaller than 1.5 Mb.

In one embodiment to the above methods, the LOH status of a bin is based on the copy number of one or more single nucleotide polymorphisms (SNPs) of interest and is determined as the mean or average of the LOH positive SNPs relative to all SNPs of interest within the bin, wherein a SNP is LOH positive or has a LOH positive status when the copy number of the minor allele (of the SNP, or comprising the SNP) is zero.

In one embodiment thereto, the (minor allele) copy number of a SNP of interest equals or is the (same as the) copy number of a segment of the (test) genomic DNA with constant (minor allele) copy number comprising the SNP of interest.

In a further or further additional embodiment to the above methods, the LOH status of a bin is based on the (minor allele) copy number of a nucleotide or nucleotide position (of interest) in the bin, and is determined as being equal to or as being the (same as the) (minor allele) copy number of a segment of the (test) genomic DNA with constant (minor allele) copy number comprising the nucleotide or nucleotide position (of interest) in the bin and wherein the bin is LOH positive or has a LOH positive status when the copy number of the minor allele of the nucleotide or nucleotide position (of interest) in the bin is zero. In particular, the nucleotide or nucleotide position (of interest) can be any nucleotide or nucleotide position in the bin (i.e. not limited to a SNP of interest). In particular, the nucleotide position (of interest) is a reference position. Furthermore in particular, the reference position in or within each bin is the same for each bin. Furthermore in particular, the reference position in or within the bin can be the center position of the bin, or can be a position within a stretch of nucleotides of or spanning 5% of the bin size on either side from the center position (e.g. if the bin size is 1 Mb, then the reference position could be within 50 kb from either side of the 500 kb midpoint of the bin).

In a further or further additional embodiment to the above methods, segments of the (test) genomic DNA with constant copy number are determined or delineated based on the copy number of a plurality of SNPs (at least 1 SNP in or within the segment and at least 1 SNP outside the segment). In a particular or particular further embodiment thereto, the plurality of SNPs is distributed over or across the (test) genomic DNA present in the sample. In a further limiting embodiment thereto, the plurality of SNPs is distributed over or across the (test) genomic DNA excluding centromeric regions. In a further particular embodiment thereto, such segment of genomic DNA with constant copy number is determined by the ASCAT algorithm or the asmultipcf algorithm (Van Loo et al. 2010, Proc Natl Acad Sci USA 107:16910-16915 or Ross et al. 2021, Bioinformatics 37:1909-1911, respectively).

Thus, in formulating the above methods, the step:
determining the LOH status of each bin, therewith providing a set of LOH/bin features can be alternatively phrased as:
determining the LOH status of each bin as the mean or average of the LOH positive SNPs relative to all SNPs within each of the bins, wherein a SNP is LOH positive or has a LOH positive status when the copy number of the minor allele (of the SNP, or comprising the SNP) is zero, therewith providing a set of LOH/bin features;
   or can be alternatively phrased as:
determining the LOH status of each bin by identifying the copy number of a nucleotide or nucleotide position (of interest) in the bin as, as being, or as being equal to/identical to/the (same as the) copy number of a segment of the genomic DNA with constant copy number comprising the nucleotide position (of interest) in the bin, and wherein a bin is LOH positive or has a LOH positive status when the copy number of the minor allele of the nucleotide or nucleotide position (of interest) in the bin is zero, therewith providing a set of LOH/bin features.

In a further alternative, the LOH status of a segment of genomic DNA with constant copy number is determined (such segment being LOH positive or having a LOH positive status when the copy number of the minor allele of the segment is zero), and the LOH status of any nucleotide or nucleotide position of interest in or within a bin (including SNPs) comprised in the segment is then defined or determined as being the (same as the), identical to, or equals the LOH status of the segment.

Any of the above methods may comprise a further step of determining or delineating segments of constant copy number in the (test) genomic DNA based on the copy number of a plurality of SNPs.

In a second aspect, the invention relates to methods for determining the loss-of-heterozygosity (LOH) status of a (test) genomic DNA sample, such methods comprising the steps of:
determining the LOH status of a plurality of single nucleotide polymorphisms (SNPs), wherein a SNP is LOH positive or has a LOH positive status when the copy number of the minor allele (of the SNP, or comprising the SNP, or of a segment of genomic DNA of constant copy number comprising the SNP) is zero;
dividing the sequence of the (test) genomic DNA in a plurality of size-defined bins (equal size(d) bins; bins defined by/of equal or same size), and determining for each (individual) bin or for each of the bins the mean or average of the LOH positive SNPs relative to all SNPs within each (individual) bin/within each bin/within each of the bins, therewith providing a set of LOH/bin features;
optionally, determining for each (individual) chromosome present in the (test) genomic DNA of the DNA sample the mean or average of the LOH positive SNPs relative to all SNPs within the chromosome/each (individual) chromosome, therewith providing a set of LOH/chromosome features;
optionally, determining for the (test) genomic DNA of the DNA sample the mean or average of the LOH positive SNPs relative to all SNPs within the (test) genomic DNA, therewith providing a LOH/genome feature;
computer-implemented data classification model/algorithm analysis of the set of LOH/bin features, and, optionally, the set of LOH/chromosome features and/or the LOH/genome feature,
   wherein the data classification model/algorithm is or was trained on a set of reference genomic DNA samples including genomic DNA samples known of being LOH positive and/or of being homologous recombination repair deficient (HRD positive) and including genomic DNA samples known of being LOH negative and/or of being homologous recombination repair proficient (HRD negative), or
   wherein the data classification model/algorithm is or was trained based on the input features comprising a set of LOH/bin features (and optionally further comprising a set of LOH/chromosome features and/or a set of LOH/genome features) of a set of reference genomic DNA samples including genomic DNA samples known of being LOH positive and/or HRD positive and including genomic DNA samples known of being LOH negative and/or HRD negative; or, alternatively,
   computer-implemented classification of the set of LOH/bin features (and optionally of the set of LOH/chromosome features and/or the LOH/genome feature) with a data classification model/algorithm (or more precisely classification of the test genomic DNA based on its set of LOH/bin features, and optionally further based on its set of LOH/chromosome features and/or the LOH/genome feature),
   wherein the data classification model/algorithm is or was trained on a set of reference genomic DNA samples (or more precisely is or was trained for classifying (as LOH or HRD positive, or as LOH or HRD negative) (a set of) reference genome DNA samples) including genomic DNA samples known of being LOH positive and/or HRD positive and including genomic DNA samples known of being LOH negative and/or HRD negative, or
   wherein the data classification model/algorithm is or was trained based on the input features comprising the (a set of) LOH/bin features (and optionally further comprising a set of LOH/chromosome features and/or a set of LOH/genome features) of a set of reference genomic DNA samples including genomic DNA samples known of being LOH positive and/or HRD positive and including genomic DNA samples known of being LOH negative and/or HRD negative;
   or, alternatively:
   computer-implemented classification, inferencing or scoring of the set of LOH/bin features (and optionally further of the set of LOH/chromosome features and/or the LOH/genome feature) of the (test) genomic DNA (or more precisely, classification, inferencing or scoring of the (test) genomic DNA based on its set of LOH/bin features, and optionally further based on its set of LOH/chromosome features and/or its LOH/genome feature) with a trained data classification algorithm or model, wherein the trained data classification algorithm or model is trained for classifying (as LOH or HRD positive, or as LOH or HRD negative) (a set of) reference genomic DNA samples including genomic DNA samples known of being LOH positive and/or HRD positive and including genomic DNA samples known of being LOH negative and/or HRD negative;
determining the (test) genomic DNA sample to be LOH positive or to have a LOH positive status when the (trained) data classification model/algorithm (or when the data classification of the above or previous step) classified, inferenced or scored the (test) genomic DNA sample as mostly/most probably/having a higher probability of/more or most closely resembling reference genomic DNA known of being LOH positive and/or HRD positive; or determining the (test) genomic DNA sample to be LOH negative or to have a LOH negative status when the (trained) data classification model/algorithm (or when the data classification of the above or previous step) classified, inferenced or scored the (test) genomic DNA sample as mostly/most probably/having a higher probability of/more or most closely resembling reference genomic DNA known of being LOH negative and/or HRD negative.

The methods of the second aspect can be alternatively defined as methods for determining the loss-of-heterozygosity (LOH) status of a (test) genomic DNA sample, such methods comprising the steps of:
determining the LOH status of a plurality of SNPs, wherein a SNP is LOH positive or has a LOH positive status when the copy number of the minor allele (of the SNP, or comprising the SNP, or of a segment of genomic DNA of constant copy number comprising the SNP) is zero;
dividing the sequence of the (test) genomic DNA of the DNA sample in a plurality of size-defined bins (equal size(d) bins; bins defined by/of equal or same size), and determining for each (individual) bin or for each of the bins the mean or average of the LOH positive SNPs relative to all SNPs within each (individual) bin/within each bin/within each of the bins, therewith providing a set of LOH/bin features;
optionally, determining for each (individual) chromosome present in the (test) genomic DNA of the DNA sample the mean or average of the LOH positive SNPs relative to all SNPs within the chromosome/each (individual) chromosome, therewith providing a set of LOH/chromosome features;
optionally, determining for the (test) genomic DNA of the DNA sample the mean or average of the LOH positive SNPs relative to all SNPs within the (test) genomic DNA, therewith providing a LOH/genome feature;
computer-implemented data classification model/algorithm analysis of the set of LOH/bin features (and optionally further of the set of LOH/chromosome features and/or the LOH/genome feature) (or more precisely analysis of the test genomic DNA based on its set of LOH/bin features and optionally based further on its set of LOH/chromosome features and/or LOH/genome feature); or, alternatively, computer-implemented classification, inferencing or scoring of the set of LOH/bin features (and optionally further of the set of LOH/chromosome features and/or the LOH/genome feature) (or more precisely of the test genomic DNA based on its set of LOH/bin features and optionally based further on its set of LOH/chromosome features and/or LOH/genome feature) with a (trained) data classification model/algorithm;
determining the (test) genomic DNA sample to be LOH positive or to have a LOH positive status when the (trained) data classification model/algorithm (or when the data classification of the above or previous step) classified, inferenced or scored the (test) genomic DNA sample as mostly/most probably/having a higher probability of/more or most closely resembling reference genomic DNA known of being LOH positive and/or HRD positive; or determining the (test) genomic DNA sample to be LOH negative or to have a LOH negative status when the (trained) data classification model/algorithm (or when the data classification of the above or previous step) classified, inferenced or scored the (test) genomic DNA sample as mostly/most probably/having a higher probability of/more or most closely resembling reference genomic DNA known of being LOH negative and/or HRD negative;
   wherein the data classification model/algorithm is or was trained on a set of reference genomic DNA samples (or more precisely is or was trained for classifying (as LOH or HRD positive, or as LOH or HRD negative) (a set of) reference genomic DNA samples) including genomic DNA samples known of being LOH positive and/or HRD positive and including genomic DNA samples known of being LOH negative and/or HRD negative, or
   wherein the data classification model/algorithm is or was trained based on the input features comprising the (a set of) LOH/bin features (and optionally further comprising the (a set of) LOH/chromosome features and/or the (a set of) LOH/genome feature) of a set of reference genomic DNA samples including genomic DNA samples known of being LOH positive and/or HRD positive and including genomic DNA samples known of being LOH negative and/or HRD negative.

In one embodiment to these methods of the second aspect, the size of the size-defined bins is smaller than 1.5 Mb.

In one embodiment to these methods of the second aspect, the data classification model or algorithm is/was trained using sets of LOH/bin features as determined for the reference genomic DNA samples.

In one embodiment these methods of the second aspect, the data classification model or algorithm is/was trained using sets of LOH/bin features and sets of LOH/chromosome features as determined for the reference genomic DNA samples.

In one embodiment to these methods of the second aspect, the data classification model or algorithm is/was trained using sets of LOH/bin features and sets of LOH/genomes feature as determined for the reference genomic DNA samples.

In one embodiment to these methods of the second aspect, the data classification model or algorithm is/was trained using sets of LOH/bin features, sets of LOH/chromosome features, and sets of LOH/genome features as determined for the reference genomic DNA samples.

In a third aspect, the invention relates to methods for determining the loss-of-heterozygosity (LOH) status of a (test) genomic DNA sample, such methods comprising the steps of:
determining the LOH status of a plurality of SNPs, wherein a SNP is LOH positive or has a LOH positive status when the copy number of the minor allele (of the SNP, or comprising the SNP, or of a segment of genomic DNA of constant copy number comprising the SNP) is zero;
dividing the sequence of the (test) genomic DNA in a plurality of size-defined bins (equal size(d) bins; bins defined by/of equal or same size), and determining for each (individual) bin or for each of the bins the mean or average of the LOH positive SNPs relative to all SNPs within each (individual) bin/within each bin/within each of the bins, therewith providing a set of LOH/bin features;
optionally, determining for each (individual) chromosome present in the (test) genomic DNA of the DNA sample the mean or average of the LOH positive SNPs relative to all SNPs within the chromosome/each (individual) chromosome, therewith providing a set of LOH/chromosome features;
optionally, determining for the (test) genomic DNA of the DNA sample the mean or average of the LOH positive SNPs relative to all SNPs within the (test) genomic DNA, therewith providing a LOH/genome feature;
classification of the set of LOH/bin features (and optionally further of the set of LOH/chromosome features and/or the LOH/genome feature) relative to a set of reference genomic DNA samples including genomic DNA samples known of being LOH positive and/or HRD positive and including genomic DNA samples known of being LOH negative and/or HRD negative; or classification, inferencing or scoring of the (test) genomic DNA based on its set of LOH/bin features (and optionally based further on the set of LOH/chromosome features and/or the LOH/genome feature), relative to a set of reference genomic DNA samples including genomic DNA samples known of being LOH positive and/or HRD positive and including genomic DNA samples known of being LOH negative and/or HRD negative;
determining the (test) genomic DNA sample to be LOH positive or to have a LOH positive status when the classification (or when the classification of the above or previous step) classified, inferenced or scored the (test) genomic DNA sample as mostly/most probably/having a higher probability of/more or most closely resembling reference genomic DNA known of being LOH positive and/or HRD positive; or determining the (test) genomic DNA sample to be LOH negative or to have a LOH negative status when the classification (or when the classification of the above or previous step) classified, inferenced or scored the (test) genomic DNA sample as mostly/most probably/having a higher probability of/more or most closely resembling reference genomic DNA known of being LOH negative and/or HRD negative;
   wherein the size of the size-defined bins is smaller than 1.5 Mb.

In one embodiment to this method of the third aspect, the classification is relative to sets of LOH/bin features as determined for the reference genomic DNA samples; or the classification is relative to classification of a set of reference genomic DNA samples based on/using sets of LOH/bin features as determined for the reference genomic DNA samples.

In one embodiment to this method of the third aspect, the classification is relative to sets of LOH/bin features and sets of LOH/chromosome features as determined for the reference genomic DNA samples; or the classification is relative to classification of a set of reference genomic DNA samples based on/using sets of LOH/bin features and sets of LOH/chromosome features as determined for the reference genomic DNA samples.

In one embodiment to this method of the third aspect, the classification is relative to sets of LOH/bin features and sets of LOH/genome features as determined for the reference genomic DNA samples; or the classification is relative to classification of a set of reference genomic DNA samples based on/using sets of LOH/bin features and sets of LOH/genome features as determined for the reference genomic DNA samples.

In one embodiment to this method of the third aspect, the classification is relative to sets of LOH/bin features, sets of LOH/chromosome features, and sets of LOH/genome features as determined for the reference genomic DNA samples; or the classification is relative to classification of a set of reference genomic DNA samples based on/using sets of LOH/bin features, sets of LOH/chromosome features, and sets of LOH/genome features as determined for the reference genomic DNA samples.

In one embodiment to any of the above methods, the plurality of single nucleotide polymorphisms (SNPs) is distributed over or across the (test) genomic DNA present in the sample. In a further embodiment to any of the above methods, the plurality of single nucleotide polymorphisms (SNPs) is distributed over or across the (test) genomic DNA excluding centromeric regions.

In one embodiment to any of the above methods of the second or third aspect, the step:
determining the LOH status of a plurality of single nucleotide polymorphisms (SNPs), wherein a SNP is LOH positive or has a LOH positive status when the copy number of the minor allele (of the SNP, or comprising the SNP, or of a segment of genomic DNA of constant copy number comprising the SNP) is zero
   can be alternatively phrased as:
determining the copy number of the minor allele for each of a plurality of single nucleotide polymorphisms (SNPs) distributed within the (test) genomic DNA present in the sample;
determining the LOH status of each SNP, wherein a SNP is LOH positive or has a LOH positive status when the copy number of the minor allele (of the SNP, or comprising the SNP, or of a segment of genomic DNA of constant copy number comprising the SNP) is zero.

In one embodiment to any of the above methods, an outcome may be that a (test) genomic DNA cannot be classified as LOH positive or negative or as having a LOH positive or negative status in case the (test) genomic DNA does not closely resemble LOH positive reference genomic DNA and does not closely resemble LOH negative reference genomic DNA. In such cases, the LOH status of the (test) genomic DNA is assigned as unknown, uncertain, not defined, undefined, or not determinable; reasons for such undefined classification include e.g. poor quality of the sample itself or a technical issue having occurred during sample preparation or sample processing.

Loss of heterozygosity (LOH) can be defined as the absence of one parent's contribution to the genetic material at a specific site in the genome. As such, a locus is LOH positive, or has LOH status, when one of the parental copies of the locus is missing, i.e. when the paternal or maternal copy of the locus is missing. A locus can be a SNP, or can comprise a SNP, or can be a genomic region comprising or covering a SNP, such as a bin referred to in the above methods. In particular, the locus or SNP is a locus or SNP of interest. In one embodiment relating to any of the above methods, LOH positive status is assigned to a SNP or locus in general when the minor allele copy number of the SNP or locus is zero—wherein LOH can be copy number neutral LOH (minor allele copy number=0; major copy number allele=2), copy number gain LOH (minor allele copy number=0; major copy number allele>2), or copy number deletion LOH (minor allele copy number=0; major copy number allele<2). With mean or average LOH as referred to herein is meant the arithmetic or mathematical mean or average of the number of SNPs or loci assessed in a defined region as having LOH positive status divided by the total number of SNPs or loci assessed in that same defined region. In the two extremes, none of the SNPs or loci are LOH positive and the mean or average LOH equals zero (0), or all of the SNPs of loci are LOH positive and the mean or average LOH equals one (1). In any of the above, “LOH positive” can be exchanged for e.g. having LOH status, or having LOH positive status; and “LOH negative” can be exchanged for e.g. not having LOH status, or having LOH negative status.

In one embodiment relating to any of the above methods, LOH as referred to herein is determined based on log ratios (log R) and B allele frequencies (BAFs). Log R refers to log-transformed copy numbers derived from sequencing depth or SNP array data; BAF to the allelic imbalance of SNPs.

In another embodiment relating to any of the above methods, and if not specified therein, the LOH status of a SNP, locus, nucleotide or nucleotide position of interest or of a region of interest (e.g. bin) is derived from the (minor allele) copy number(s) of one or more segments of the genomic DNA determined to have a constant copy number (within the segment), wherein the segment is comprising the SNP, locus, nucleotide or nucleotide position of interest or is comprising or in part comprising the region (e.g. bin) of interest.

In yet a further particular embodiment relating to any of the above methods, and if not specified therein, the LOH status of a segment of the genomic DNA, comprising the SNP, locus, nucleotide or nucleotide position of interest or comprising or in part comprising the region (e.g. bin) of interest, is determined, and the LOH status of the SNP, locus, nucleotide or nucleotide position of interest or of a region of interest (e.g. bin) equals or is the same as or is identical to the LOH status of the segment of genomic DNA. In a further particular embodiment thereto, such segment of genomic DNA of/having a constant copy number is a segment as determined by the ASCAT algorithm or the asmultipcf algorithm (Van Loo et al. 2010, Proc Natl Acad Sci USA 107:16910-16915 or Ross et al. 2021, Bioinformatics 37:1909-1911, respectively). Such segments can be delineated based on the copy number determination of a plurality of SNPs, loci, nucleotides or nucleotide positions, such as a plurality of SNPs, loci, nucleotides or nucleotide positions distributed across the genome. In particular the distribution of the SNPs, loci, nucleotides or nucleotide positions across the genome is such that a full genome is covered, with the possible exception of centromere regions. A further possible exception are the sex chromosomes that can optionally be excluded from any analysis.

An advantage of reducing the size of the regions of interest (e.g. bins) is that the copy number of such region of interest (e.g. bins) can be reliably determined based on the copy number as determined for the segments of genomic DNA with constant copy number: the smaller the size of the region of interest (e.g. bin), the larger the chance that there will be no switch of copy number within the region of interest. As such, the copy number of a small enough region of interest (e.g. bin) will with very few exceptions be identical to, be equal to, or be the same as the copy number of a segment of genomic DNA with constant copy number. This then logically extrapolates to all SNPs, loci, nucleotides or nucleotide positions of interest present within the region of interest (e.g. bin). Deriving copy number of an individual SNP, locus, nucleotide or nucleotide position of interest from a segment of genomic DNA with constant copy number comprising the individual SNP, locus, nucleotide or nucleotide position of interest may additionally reduce possible noise-induced errors occurring by individual determination of the copy number of the SNP, locus, nucleotide or nucleotide position of interest.

Therefore, in one embodiment, any of the above methods may comprise a step of determining or delineating segments of constant copy number in the (test) genomic DNA based on the copy number of a plurality of SNPs, loci, nucleotides or nucleotide positions.

Therefore in one embodiment to any of the methods of the above second and third aspects, the step:
determining the LOH status of a plurality of single nucleotide polymorphisms (SNPs), wherein a SNP is LOH positive or has a LOH positive status when the copy number of the minor allele (of the SNP, or comprising the SNP, or of a segment of genomic DNA of constant copy number comprising the SNP) is zero
   can be alternatively phrased as:
determining or delineating segments of constant copy number in the (test) genomic DNA based on the copy number of a plurality of SNPs;
determining the LOH status of the plurality of single nucleotide polymorphisms (SNPs), wherein a SNP is LOH positive or has a LOH positive status when the copy number (of the minor allele) of the segment of constant copy number comprising the SNP is zero.

In a further alternative, the LOH status of a segment of genomic DNA with constant copy number is determined (such segment being LOH positive or having a LOH positive status when the copy number of the minor allele of the segment is zero), and the LOH status of any nucleotide position of interest in or within a bin (including SNPs) comprised in the segment is then the (same as the), identical to, or equals the LOH status of the segment.

In a further alternative embodiment to any of the above methods of the above second and third aspects, the steps:
determining the LOH status of a plurality of single nucleotide polymorphisms (SNPs), wherein a SNP is LOH positive or has a LOH positive status when the copy number of the minor allele (of the SNP, or comprising the SNP, or of a segment of genomic DNA of constant copy number comprising the SNP) is zero;
dividing the sequence of the (test) genomic DNA in a plurality of size-defined bins (equal size(d) bins;
   bins defined by/of equal or same size), and determining for each (individual) bin or for each of the bins the mean or average of the LOH positive SNPs relative to all SNPs within each (individual) bin/within each bin/within each of the bins, therewith providing a set of LOH/bin features can be alternatively phrased as:
determining or delineating segments of constant copy number in the (test) genomic DNA based on the copy number of a plurality of SNPs, loci, nucleotides or nucleotide positions;
dividing the sequence of the (test) genomic DNA in a plurality of size-defined bins (equal size(d) bins;
   bins defined by/of equal or same size);
determining for each (individual) bin or for each of the bins the LOH status by identifying the (minor allele) copy number of a SNP, locus, nucleotide or nucleotide position (of interest) in the bin as, as being, or as being equal to/identical to/the (same as the) (minor allele) copy number of a segment of the genomic DNA with constant copy number comprising the SNP, locus, nucleotide or nucleotide position (of interest) in the bin, and wherein a bin is LOH positive or has a LOH positive status when the copy number of the minor allele of the SNP, locus, nucleotide or nucleotide position (of interest) in the bin is zero, therewith providing a set of LOH/bin features. In one embodiment to this step, the SNP, locus, nucleotide or nucleotide position (of interest) in or within a bin is the same nucleotide position in each bin. Further in particular, the SNP, locus, nucleotide or nucleotide position (of interest) in the bin can be a reference SNP, locus, nucleotide or nucleotide position in or within the bin. In particular, such reference SNP, locus, nucleotide or nucleotide position can be the center position of the bin, or can be a position within a stretch of nucleotides of 5% of the bin size on either side from the center position (e.g. if the bin size is 1 Mb, then the reference position could be within 50 kb from either side of the 500 kb midpoint of the bin). In a further alternative, the LOH status of a segment of genomic DNA with constant copy number is determined (such segment being LOH positive or having a LOH positive status when the copy number of the minor allele of the segment is zero), and the LOH status of any SNP, locus, nucleotide or nucleotide position of interest in or within a bin (including the SNP, locus, nucleotide or nucleotide position of interest) comprised in the segment is then the (same as the), identical to, or equals the LOH status of the segment.

In a further embodiment relating to any of the above methods, the set of LOH/bin features is maintained during classification or during computer-implemented data classification, meaning that individual features of the set are not combined, merged, added or collapsed into a larger bin (with the exception of the optional separate LOH/chromosome and LOH/genome features). Thus individual features of the set of LOH/bin features are not combined, merged, added or collapsed into a bin larger than a single bin but smaller than a full chromosome or smaller than the genome.

In a further embodiment relating to any of the above methods, centromere regions of chromosome(s) are excluded from the set of LOH/bin features and the optional separate LOH/chromosome and LOH/genome features.

In a further embodiment relating to any of the above methods, sex chromosomes (X-chromosome and/or Y-chromosome) are excluded from the set of LOH/bin features and the optional separate LOH/chromosome and LOH/genome features.

In another embodiment relating to any of the above methods, the copy number of a SNP, locus, nucleotide or nucleotide position of interest is determined based on, or starting from, sequencing of the (test or reference) genomic DNA comprising the SNP, locus, nucleotide or nucleotide position.

In one particular embodiment, the sequencing is targeted (e.g. involving linear, non-linear or PCR amplification and/or capture by hybridization of a genomic DNA region comprising a SNP or locus of interest) or untargeted (e.g. whole genome, exome, transcriptome) sequencing. Nucleotide sequencing is possible by any known means including classical Sanger sequencing, massive parallel sequencing and (nano)pore sequencing.

In a further particular embodiment, the sequencing is at low coverage, at high coverage, at 1× to 10× coverage, at 1× to 500× coverage, at 1× coverage, at 20× coverage, at 30× coverage, at 40× coverage, is at 50× to 500× coverage, is at 50× coverage, at 60× coverage, at 70× coverage, at 80× coverage, at 90× coverage, at 100× coverage, at 110× coverage, at 120× coverage, at 130× coverage, at 140× coverage, at 150× coverage, at 160× coverage, at 170× coverage, at 180× coverage, at 190× coverage, or at 200× coverage; or wherein the sequencing is at 50× to 1000× coverage, at 300× coverage, at 400× coverage, at 500× coverage, at 600× coverage, at 700× coverage, at 800× coverage, at 900× coverage, or at 1000× coverage. Alternatively, the sequencing is shallow sequencing, deep sequencing, ultradeep sequencing, or maximum-depth sequencing.

In a further particular embodiment, the sequencing is paired-end sequencing such as 2×50 bp reads to 2×200 bp reads (e.g. 2×50 bp reads, 2×60 bp reads, 2×70 bp reads, 2×80 bp reads, 2×90 bp reads, 2×100 bp reads, 2×110 bp reads, 2×120 bp reads, 2×130 bp reads, 2×140 bp reads, 2×150 bp reads, 2×151 bp reads, 2×160 bp reads, 2×170 bp reads, 2×180 bp reads, 2×190 bp reads, 2×200 bp reads). In particular, the sequenced stretch of the genomic DNA is comprising one or more of the plurality of SNPs, loci, nucleotides or nucleotide positions of interest.

In a further particular embodiment, the sequencing reads are aligned to a reference genome (such as the human hg18, hg19, NCBI/hg18, GRCh37/hg19, or GRCh38.p14 reference genome).

In a further particular embodiment, the copy number of a SNP or locus of interest is determined based on, or starting from, minimum quality sequencing data (e.g. minimum base quality and/or minimum mapping quality).

In a further particular embodiment, determination of the copy number of a SNP, locus, nucleotide or nucleotide position of interest is involving an algorithm or bio-informatic algorithm such as run or executed on a computer. Such algorithm can e.g. be based on a hidden Markov algorithm or on circular binary segmentation.

In a further particular embodiment, determination of the copy number of a SNP, locus, nucleotide or nucleotide position of interest is involving an analytical method compensating for contamination present in the DNA sample (e.g. if the DNA sample is obtained from a tumor sample, DNA from non-tumor cells is a “contaminant” expected to be present in the DNA sample). Numerous methods and kits are available enabling extraction of DNA from cells.

In a further particular embodiment to any of the above methods, the SNP, locus, nucleotide or nucleotide position of interest, or the genomic DNA comprising a SNP, locus, nucleotide or nucleotide position of interest is captured from the genomic DNA sample by means of (hybridization to) an oligonucleotide probe. Alternatively, a plurality of SNPs, loci, nucleotides or nucleotide positions of interest, or a plurality of SNPs, loci, nucleotides or nucleotide positions of interest-comprising genomic DNA molecules, is captured by or captured by means of (hybridization to) a library of oligonucleotide probes (capture library/nucleic acid capture), or captured by or captured by means of (hybridization to) a plurality of oligonucleotide probes. This process is also known as hybridization capture or target enrichment. The process may include prior shearing, such as mechanical shearing, of the input material (e.g. DNA extracted from formalin fixed paraffin embedded (FFPE) tumor sample). The genomic DNA sample from a tumor or cancer cell may in principle be any type of biological sample comprising tumor genomic DNA including fresh or processed (e.g. FFPE, fresh frozen) tumor biopsy samples, circulating tumor DNA, genomic DNA from circulating tumor cells etc. The tumor or cancer can be of primary origin or of metastatic origin. The tumor or cancer may in particular be a tumor or cancer known to be prone or susceptible to (genetic scarring by means of) any defect or impairment in the homologous recombination repair pathway; or known to be prone or susceptible to losing homologous recombination repair proficiency (or HRD negative status); or known to be prone or susceptible to become homologous recombination repair deficient (or HRD positive). The tumor or cancer may in particular be any of e.g. ovarian, breast, colon, prostate, pancreatic, lung, renal or esophagal origin. A plurality of SNPs, loci, nucleotides or nucleotide positions is meant to include more than 1 SNP, locus, nucleotide or nucleotide position, thus 2 or more SNPs, loci, nucleotides or nucleotide positions. In particular, between 2 and 200000 (200 k) SNPs, loci, nucleotides or nucleotide positions are envisaged, or between 2 and 150 k, between 2 and 100 k, between 2 and 75 k, between 2 and 50 k, or between 2 and 25 k SNPs, loci, nucleotides or nucleotide positions; or between 10 k and 200 k SNPs, loci, nucleotides or nucleotide positions, or between 10 k and 150 k, or between 10 k and 100 k, or between 10 k and 75 k, or between 10 k and 50 k, or between 10 k and 25 k SNPs, loci, nucleotides or nucleotide positions.

In a particular embodiment thereto, such oligonucleotide probe(s) are attached to a solid support such as a sheet, bead, or plate well (of any suitable size or dimension). In a particular embodiment thereto, a spacer is introduced between the solid support and the oligonucleotide probe(s) to support efficient capture of the DNA of interest from the sample such as biological sample.

In a particular embodiment thereto, the individual oligonucleotide probes can have a length of 50 to 150 bp, or an average length of 50 bp, 60 bp, 70 bp, 80 bp, 90 bp, of 100 bp, of 110 bp, of 120 bp, of 130 bp, of 140 bp, or of 150 bp.

In a particular embodiment thereto, the capture library is optionally comprising or further comprising one or a set of gene-specific oligonucleotide probes.

In a further particular embodiment, the capture oligonucleotides comprise molecular barcodes, molecular indexes, or unique molecular identifiers (UMIs).

In a further particular embodiment to any of the above methods, the size of the size-defined bins is lower than/smaller than 1500 kb(p) (1.5 Mb(p)), e.g. about 50 kb(p) to 1400 kb(p), e.g. approximately 50 kb(p), e.g. approximately 100 kb(p), e.g. approximately 200 kb(p), e.g. approximately 300 kb(p), e.g. approximately 400 kb(p), e.g. approximately 500 kb(p), approximately 550 kb(p), approximately 600 kb(p), approximately 650 kb(p), approximately 700 kb(p), approximately 750 kb(p), approximately 800 kb(p), approximately 850 kb(p), approximately 900 kb(p), approximately 950 kb(p), approximately 1000 kb(p), approximately 1050 kb(p), approximately 1100 kb(p), approximately 1150 kb(p), approximately 1200 kb(p), approximately 1250 kb(p), approximately 1300 kb(p), approximately 1350 kb(p), approximately 1400 kb(p), approximately 1450 kb(p); and wherein the size of one or more bins may deviate from the size of the majority of the bins depending on chromosome size (e.g. left-over sequence smaller than chosen bin-size after dividing a chromosome or chromosome-arm in bins of the chosen size). A size-defined bin can cover either no SNP, locus, nucleotide or nucleotide position of interest, one SNP, locus, nucleotide or nucleotide position of interest, two SNPs, loci, nucleotides or nucleotide positions of interest, or three or more SNPs, loci, nucleotides or nucleotide positions of interest.

The terms “computer-implemented data classification model/algorithm analysis” or “computer-implemented classification with a data classification algorithm” are used herein interchangeably and refer to use of a machine-learning based data classification model or algorithm or to use of a machine-learning model or algorithm for data classification. In particular in the current context, determination of the LOH status of a test genomic DNA sample can involve an algorithm or bio-informatic algorithm such as run or executed on a computer; in particular the algorithm is a machine learning algorithm or model, more in particular the algorithm is a data classification algorithm or data classifier algorithm. Many data classification algorithms exist and include Random Forest (RF), Support Vector Machines (SVM), K-Nearest Neighbors (KNN), and Linear Discriminant Analysis (LDA) (as explained in e.g. Chen et al. 2020, J Big Data 7, 52), and further algorithms involving regression or logistic regression analysis, gradient boosting based analysis, analyses based on multi layer perceptron (MLP), decision trees (DT), adaboost (Ada) (Turgut et al. 2018 doi: 10.1109/EBBT.2018.8391468), (nearest) shrunken centroid classifier algorithms, etc.

Prior to determining the status of interest of a test item with the help of a computer-implemented data classification algorithm or model, the machine learning model or algorithm must be trained with a set of training items of which the status of interest is known, or must be trained with a set of reference items. In particular in the current context, a set of reference genomic DNA samples was used. More in particular, a set of reference genomic DNA samples of an exemplary clinical trial study was used, wherein the exemplary clinical trial studied the effect of adding the PARP inhibitor olaparib to bevacizumab therapy of ovarian cancer, the beneficial effect of such addition being more pronounced/substantial in patients with a HRD-positive tumor. More in particular, the clinical trial is the PAOLA-1 trial (Ray-Coquard et al. 2019, N Engl J Med 381: 2416-2428; ClinicalTrials.gov identifier NCT02477644). More in particular, the data classification model or algorithm is trained using (as input features) sets of LOH/bin features (and optionally further sets of LOH/chromosome features, and/or sets of LOH/genome features) as determined for the reference genomic DNA samples (comprising genomic DNA samples known of being LOH positive and/or HRD positive and genomic DNA samples known of being LOH negative and/or HRD negative).

Alternatively, classification based on the sets of LOH/bin features (and optionally further of the sets of LOH/chromosome features, and/or the LOH/genome feature) as determined for a (test) genomic DNA sample is done without a dedicated (computer-implemented) data classification model or data classification algorithm. Indeed, e.g. all individual features can be mapped and such map can be compared to a corresponding map of a reference genomic DNA. When such map of a (test) genomic DNA sample is more or most closely resembling a reference map of LOH positive (or negative) reference genomic DNA, then the (test) genomic DNA can be classified as LOH positive (or negative). Such mapping obviously may involve use of a software application or program run on a computer (without the software classifying the data).

In a particular embodiment to any of the above methods, a reference genomic DNA sample is assigned as being LOH positive, or as being LOH negative, based on a method, kit or assay known or available in the art. In particular LOH status of a reference genomic DNA sample can have been determined by means of a HRD-LOH score. It can alternatively have been determined by means of score based on and including HRD-LOH status, large scale transition (LST) status and telomeric allelic imbalance (TAI) status (such as in the Myriad®myChoice® test), combined leading to the determination of a HRD positive (homologous recombination repair deficient) status or a HRD negative (homologous recombination repair proficient) status of a DNA sample. The HRD-LOH score in particular is based on the number of LOH regions of intermediate size (longer than 15 Mb but shorter than the whole chromosome: Abkevich et al. 2012, Br J Cancer 107:1776-1782) occurring in the DNA of a tumor sample. Large-scale state transitions (LST) are chromosomal breaks between adjacent genomic regions longer than 10 Mb that are increased in BRCA1/2 mutant cancers (Popova et al., Cancer Res 72:5454-5462). Telomeric-allelic imbalance (TAI) is allelic imbalance extending to the subtelomere but not crossing the centromere and is enriched in cancers with BRCA1/2 deficiency and response to platinum chemotherapy (Birkbak, et al. 2012, Cancer Discov 2:366-375). As confirmed in the Examples herein, fractional LOH or percentage LOH are parameters that are less reliable in determining LOH status of a (test or) reference genomic DNA sample; this is in line with previous observations reported by Stover et al. 2020 (Gynecologic Oncology 159:887-898) such that 19%-61% of patients identified as positive by myChoice® HRD would have been missed by % LOH. Based on a set of LOH and/or HRD positive and -negative reference genomic DNA samples, an LOH positive and -negative reference genomic DNA profile can be made/is made by means of classification (such as by means of machine learning assisted classification) as is subject of this disclosure.

A test genomic DNA sample in general will have an LOH status profile more or most closely resembling the LOH positive and/or HRD positive reference genomic DNA profile or more or most closely resembling the LOH negative and/or HRD negative reference genomic DNA profile and can that way be classified as having either LOH positive status or LOH negative status. An alternative outcome may be that a (test) genomic DNA cannot be classified as LOH positive or negative or as having a LOH positive or negative status in case the (test) genomic DNA does not closely resemble LOH positive reference genomic DNA and does not closely resemble LOH negative reference genomic DNA. In such cases, the LOH status of the (test) genomic DNA is assigned as unknown, uncertain, not defined, undefined, or not determinable (possible causes: see above).

In a further aspect of the invention, any of the above-mentioned methods are methods to determine the homologous recombination deficiency (HRD) status of a tumor. In particular, any of the above-mentioned methods are methods for determining the homologous recombination deficiency (HRD) status of the genomic DNA sample, wherein a genomic DNA sample classified as being LOH positive or as having a LOH positive status is HRD positive or is having a HRD positive status; or, wherein a genomic DNA sample classified as being LOH negative or as having a LOH negative status is HRD negative or is having a HRD negative status. It has indeed been shown before that the overall LOH status of tumor-associated DNA is a footprint linked to defects in a cell's homologous recombination repair machinery, such as defects in the homologous recombination repair genes BRCA1 and/or BRCA2. In particular, one of the initial HRD scores was build based on the number of LOH regions of intermediate size (longer than 15 Mb but shorter than the whole chromosome: Abkevich et al. 2012, Br J Cancer 107:1776-1782) occurring in the DNA of a tumor sample, and is sometimes referred to as the “HRD-LOH” score. It is thus accepted in the field that determining LOH status is a proxy or alternative for determining HRD-status.

As defects in the homologous recombination repair genes BRCA1 and/or BRCA2 are a major driver of HRD, any of the above-mentioned methods are alternatively methods to determine defects in the homologous recombination repair genes BRCA1 and/or BRCA2 wherein a LOH positive status or HRD positive status of the analyzed genomic DNA is indicative of the presence of a defect in the BRCA1 and/or BRCA2 gene, or is indicative of a BRCA positive status (defects present—as opposed to a BRCA negative status when no defects are present); or, wherein a genomic DNA sample classified as being LOH or HRD negative or as having a LOH or HRD negative status is BRCA negative or is having a BRCA negative status. In one embodiment to these methods, an outcome may be that a (test) genomic DNA cannot be classified as LOH positive or negative or as having a LOH positive or negative status in case the (test) genomic DNA does not closely resemble LOH positive reference genomic DNA and does not closely resemble LOH negative reference genomic DNA. In such cases, the HRD or BRCA status of the (test) genomic DNA is assigned as unknown, uncertain, not defined, undefined, or not determinable (possible causes: see above).

The presence of HRD in a tumor cell furthermore is at the basis of identifying cancer patients that are expected to respond well to therapies (further) inhibiting or impairing DNA repair or otherwise damaging DNA, such as with inhibitors of poly ADP ribose polymerase (PARP)(e.g. olaparib, rucaparib, niraparib, talazoparib, veliparib, pamiparib), of type I topoisomerases (e.g. camptothecins such as topotecan, irinotecan and belotecan; and non-camptothecins such as indenoisoquinoline, phenthridines and indolocarbazoles), of type II topoisomerases (e.g. doxorubicin, daunorubicin, and other anthracycline antibiotics (also acting as DNA damaging agent by intercalating in DNA)), of dihydrofolate reductase (DHFR; e.g. methotrexate) or with DNA damaging agents or treatments. DNA damaging treatments include ionizing radiation, irradiation, radiotherapy, or UV radiation. DNA damaging agents further include platins or platinum-containing chemotherapeutics/coordination complexes of platinum, such as cisplatin, carboplatin, oxaliplatin, nedaplatin, triplatin tetranitrate, phenanthriplatin, picoplatin, satraplatin (overall also referred to as platinum-based agents). DNA damaging agents yet further non-exhaustively include alkylating agents (e.g. cyclophosphamide), antimetabolites (e.g. 5-fluorouracil, capecitabine, floxuridine, gemcitabine; purine analogs such as 6-mercaptopurine, 8-azaguanine, fludarabine, and cladribine) and cytotoxic antibiotics (e.g. bleomycin).

Any of the above methods may optionally comprise a clinician or a medical professional (e.g. nurse or doctor) taking or obtaining a tumor sample or sample comprising a tumor or cancer cell from a subject or patient; and/or a clinician, medical professional, laboratory technician or laboratory professional obtaining or isolating the genomic DNA from a tumor sample or sample comprising a tumor or cancer cell; and/or a clinician, medical professional, laboratory technician or laboratory professional determining or assessing the LOH, HRD and/or BRCA status of the genomic DNA. In particular, General Data Protection Regulations (GDPRs) are complied with.

Any of the above methods may optionally comprise a clinician or a medical professional (e.g. nurse or doctor), or laboratory technician or laboratory professional, providing a tumor sample or sample comprising a tumor or cancer cell (obtained from a subject or patient) directly or indirectly to a test laboratory (such as independent accredited test laboratory); and/or a clinician or a medical professional, or laboratory technician or laboratory professional, providing the genomic DNA from a tumor sample or sample comprising a tumor or cancer cell (obtained from a subject or patient) directly or indirectly to a test laboratory (such as independent accredited test laboratory); and/or a clinician or a medical professional (e.g. nurse or doctor), or laboratory technician or laboratory professional receiving (in tangible or intangible form) the LOH, HRD and/or BRCA status of the genomic DNA as determined or assessed by a test laboratory (such as independent accredited test laboratory); wherein such test laboratory (such as independent accredited test laboratory; or one or its technicians or professionals) is able to perform or is capable of performing a method of determining LOH, HRD and/or BRCA status as described hereinabove, or is able of or capable of performing an analysis with a diagnostic kit as described hereinafter. In particular, GDPRs are complied with.

Another aspect of the invention relates to methods of predicting the response of a subject or patient having cancer or a tumor to a treatment or treatment regimen comprising a DNA damaging agent or therapy and/or comprising an agent inhibiting or impairing DNA repair or therapy comprising inhibiting or impairing DNA repair, wherein the subject or patient (having the cancer or tumor) is likely to respond to the treatment or treatment regimen if a genomic DNA sample obtained from the tumor or from a cancer cell of the cancer or tumor is determined to have a LOH positive, HRD positive and/or BRCA positive status according to any of the relevant methods described hereinabove. In an alternative, such methods are methods for determining or assessing if a subject or patient is likely to respond to such treatment or treatment regimen; or are methods for determining or assessing the likelihood of response of a subject or patient to such treatment or treatment regimen; or are methods predicting a likely response of a subject or patient to such treatment or treatment regimen. More in particular, such methods comprise determining the LOH, HRD, or BRCA-status of a test genomic DNA sample obtained from the subject or patient according to, with, via, or applying any of the above methods, and predicting the subject or patient to respond, or likely to respond, to the treatment, treatment regimen, or therapy when the test genomic DNA is determined to be LOH, HRD, or BRCA positive or to have a positive LOH, HRD, or BRCA status. If in such methods the test genomic DNA is determined to be LOH, HRD, or BRCA negative or to have a negative LOH, HRD, or BRCA status, the methods are then predicting the subject or patient not to respond, or likely not to respond, to the treatment, treatment regimen, or therapy. It is a plausible assumption that response to therapy with one member of a class of therapeutic agents can be extrapolated to response to therapy with another member of the same class of therapeutic agents. For example, response to therapy with an agent inhibiting DNA repair (e.g. an inhibitor of PARP) will plausibly be similar to response to therapy with a DNA damaging agent (e.g. a platinum-based or platinum-containing agent) as both agents provoke the same effect i.e. DNA damage. Such methods may optionally comprise a clinician or a medical professional (e.g. nurse or doctor) making such prediction or assessment after having determined the LOH, HRD and/or BRCA status of the genomic DNA or after having received (in tangible or intangible form) the LOH, HRD and/or BRCA status of the genomic DNA as determined by a test laboratory (cf. supra). Such methods may optionally comprise a test laboratory (cf. supra) making such prediction or assessment after having determined the LOH, HRD and/or BRCA status of the genomic DNA, or after having received (in tangible or intangible form) the LOH, HRD and/or BRCA status of the genomic DNA as determined by an individual or entity not linked to the test laboratory.

The invention in a further aspect relates to a DNA damaging agent or therapy comprising a DNA damaging agent, and/or to an agent inhibiting or impairing DNA repair or therapy comprising an agent inhibiting or impairing DNA repair—or to a treatment or treatment regimen comprising a DNA damaging agent and/or comprising an agent inhibiting or impairing DNA repair—for use in (the manufacture of a medicament for) treating a tumor or cancer if a genomic DNA sample obtained from the tumor or from a cancer cell of the cancer or tumor is determined to have a LOH positive, HRD positive and/or BRCA positive status according to any of the relevant methods described hereinabove. More in particular, the DNA damaging agent or therapy comprising the DNA damaging agent, and/or the agent inhibiting or impairing DNA repair or therapy comprising the agent inhibiting or impairing DNA repair, or the treatment or treatment regimen comprising a DNA damaging agent and/or comprising an agent inhibiting or impairing DNA repair are for use in (the manufacture of a medicament for) treating a (subject having a) tumor or cancer, comprising
determining the LOH, HRD, or BRCA status of a genomic DNA sample obtained from the tumor or from a cancer cell of the cancer or tumor, according to any of the above-described methods;
administering the agent to the subject if the LOH, HRD, or BRCA status of the genomic DNA sample is positive (as determined in the previous step).

Alternatively, the invention relates to methods for treating a subject or patient having cancer or a tumor, such methods comprising administering a DNA damaging agent or therapy comprising a DNA damaging agent to the subject or patient having the cancer or tumor, and/or administering an agent inhibiting or impairing DNA repair or therapy comprising an agent inhibiting or impairing DNA repair to the subject or patient having the cancer or tumor, if a genomic DNA sample obtained from the tumor or from a cancer cell of the cancer or tumor is determined to have a LOH positive, HRD positive and/or BRCA positive status according to any of the relevant methods described hereinabove.

Alternatively, the invention relates to methods for treating a subject or patient having cancer or a tumor, such methods comprising:
determining a genomic DNA sample obtained from the tumor or from a cancer cell of the cancer or tumor to have a LOH positive or HRD positive status according to any of the relevant methods described hereinabove; and
administering a DNA damaging agent or therapy comprising a DNA damaging agent to the subject or patient having the cancer or tumor, and/or administering an agent inhibiting or impairing DNA repair to the subject or patient having the cancer or tumor, and/or administering a therapy comprising an agent inhibiting or impairing DNA repair to the subject or patient having the cancer or tumor.

By administering the DNA damaging agent (or therapy comprising it) and/or the agent inhibiting or impairing DNA repair (or therapy comprising it) to the subject or patient having the cancer or tumor, the subject or patient is being treated. In particular, a therapeutically effective amount or dose of the DNA damaging agent and/or agent inhibiting or impairing DNA repair is administered to the subject or patient as (part of a) treatment or as part of a treatment regimen. When the DNA damaging agent and/or agent inhibiting or impairing DNA repair is combined with a further anticancer agent, then the amount or dose of the DNA damaging agent and/or agent inhibiting or impairing DNA repair may in itself not be sufficient to result in treatment, but then is sufficient in combination with the further anticancer agent. Such combinations of treatment modalities may e.g. decrease possible adverse effects or side effects of (one of) the individual modalities.

“Treatment”/“treating” refers to any rate of reduction, delaying or retardation of the progress of the disease or disorder, or a single symptom thereof, compared to the progress or expected progress of the disease or disorder, or single symptom thereof, when left untreated. This implies that a therapeutic modality on its own may not result in a complete or partial response (or may even not result in any response), but may, in particular when combined with other therapeutic modalities (such as, but not limited thereto: surgery, radiation, etc.), contribute to a complete or partial response (e.g. by rendering the disease or disorder more sensitive to therapy). More desirable, the treatment results in no/zero progress of the disease or disorder, or single symptom thereof (i.e. “inhibition” or “inhibition of progression”), or even in any rate of regression of the already developed disease or disorder, or single symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

A “therapeutically effective amount or dose” refers to an amount of a therapeutic agent to treat, inhibit or prevent a disease or disorder in a subject (such as a mammal). In the case of cancers, the therapeutically effective amount of the therapeutic agent may reduce the number of cancer cells; reduce the primary tumor size; inhibit (i.e., slow down to some extent and preferably stop) cancer cell infiltration into peripheral organs; inhibit (i.e., slow down to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, e.g., be measured by assessing the duration of survival (e.g. overall survival), time to disease progression (TTP), response rates (e.g., complete response and partial response, stable disease), length of progression-free survival (PFS), duration of response, and/or quality of life.

The term “effective amount (or dose)” or “therapeutically effective amount (or dose)” may depend on the dosing regimen of the agent/therapeutic agent or composition comprising the agent/therapeutic agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses. The effective amount may further vary depending on the severity of the condition that needs to be treated; this may depend on the overall health and physical condition of the subject or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a subject (such as a mammal) in need thereof, i.e., harboring a tumor, cancer or neoplasm in need of treatment. In general a (therapeutically) effective amount of (a) therapeutic compound(s) is administered to the mammal in need thereof in order to obtain the described clinical response(s).

“Administering” means any mode of contacting that results in interaction between an agent (e.g. a therapeutic compound or immunotherapeutic compound or agent) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

Another aspect of the invention relates to methods of prognosing survival or determining or assessing survival probability of a subject or patient having cancer or a tumor upon receiving treatment or a treatment regimen comprising a DNA damaging agent or therapy and/or comprising an agent inhibiting or impairing DNA repair or therapy comprising inhibiting or impairing DNA repair, wherein the subject or patient (having the cancer or tumor) is likely to have an increased survival or increased survival probability if a genomic DNA sample obtained from the tumor or from a cancer cell of the cancer or tumor is determined to have a LOH positive, HRD positive and/or BRCA positive status according to any of the relevant methods described hereinabove; or, wherein the subject or patient (having the cancer or tumor) is likely to have an decreased survival or decreased survival probability if a genomic DNA sample obtained from the tumor or from a cancer cell of the cancer or tumor is determined to have a LOH negative, HRD negative and/or BRCA negative status according to any of the relevant methods described hereinabove. More in particular, such methods comprise determining the LOH-, HRD-, or BRCA-status of a test genomic DNA sample obtained from the subject or patient according to, with, via, or applying any of the above methods, and prognosing, determining or assessing the subject or patient receiving treatment or a treatment regimen comprising a DNA damaging agent or therapy and/or comprising an agent inhibiting or impairing DNA repair or therapy comprising inhibiting or impairing DNA likely to have an increased survival or increased survival probability upon repair when or if the test genomic DNA is determined to be LOH, HRD, or BRCA positive or to have a positive LOH, HRD, or BRCA status. If or when in such methods the test genomic DNA is determined to be LOH, HRD, or BRCA negative or to have a negative LOH, HRD, or BRCA status, the methods are then prognosing, determining or assessing the subject or patient receiving treatment or a treatment regimen comprising a DNA damaging agent or therapy and/or comprising an agent inhibiting or impairing DNA repair or therapy comprising inhibiting or impairing DNA likely not to have an increased survival or increased survival probability upon receiving treatment or a treatment regimen.

Such increased survival or increased survival probability is a consequence of the increased likelihood of response of the subject or patient to the treatment or treatment regimen. Such increased survival or increased survival probability is in comparison with survival or survival probability of a reference subject or patient, such as the average survival or survival probability of subjects or patients with the same tumor or cancer having a LOH positive, HRD positive and/or BRCA positive status but not receiving the treatment or treatment regimen, or such as the average survival or survival probability of subjects or patients with the same tumor or cancer having a LOH negative, HRD negative and/or BRCA negative status either receiving or not receiving the treatment or treatment regimen. Survival can be expressed as progression-free survival, relapse-free survival, overall survival, etc., and can be determined e.g. by the Kaplan-Meier method or analysis.

In one embodiment to these methods, an outcome may be that a (test) genomic DNA cannot be classified as LOH positive or negative or as having a LOH positive or negative status in case the (test) genomic DNA does not closely resemble LOH positive reference genomic DNA and does not closely resemble LOH negative reference genomic DNA. In such cases, a prognosis of survival or survival probability cannot be defined, determined or assessed with reasonable certainty.

Any such methods may optionally comprise a clinician or a medical professional (e.g. nurse or doctor), or a test laboratory (cf. supra) determining the survival probability.

Any of the above methods in one particular embodiment are in vitro methods, or methods on biologicals samples having been obtained from a subject or patient. In a further particular embodiment, the subject or patient is a treatment-naïve subject or patient. In any of the above, a subject or patient in general is a mammalian species having cancer or a tumor or diagnosed with cancer. The mammalian species in general is a higher species including primates, cattle (e.g. cows, sheep, goats, pigs), horses, and pets (e.g. dogs, cats). In one embodiment the subject or patient is a human subject or patient.

The invention further relates to systems, such as computer systems, data-processing systems, computer or machine readable media, or diagnostic kits for use in performing any of the above-mentioned methods. In one embodiment, such (computer/data-processing) systems or kits are comprising a (trained) data classification algorithm, or a computer or machine readable medium comprising a (trained) data classification algorithm; or are comprising a computer program for execution of a data classification algorithm or model; wherein the data classification algorithm is trained to classify a test genomic DNA sample as being LOH, HRD, and/or BRCA positive, or as being LOH, HRD and/or BRCA negative. In particular, the data classification algorithm is trained with input features or based on input features comprising sets of LOH/bin features (and optionally further comprising sets of LOH/chromosome features and/or sets of LOH/genome features) as described hereinabove. Furthermore in particular, the data classification algorithm is, using the input features, trained on a set of reference genomic DNA samples including genomic DNA samples known of being LOH positive and/or HRD positive and genomic DNA samples known of being LOH negative and/or HRD negative as described hereinabove. Alternatively, such (diagnostic) kits are comprising a set of immobilized oligonucleotides suitable for capturing from a genomic DNA sample the genomic DNA required for the determination of the LOH/bin features (and optionally, the LOH/chromosome features and/or the LOH/genome feature); in particular, the number of immobilized oligonucleotides is ranging from 10 k to 200 k.

Related to computer programs, this disclosure covers any computer program that in particular is having the instructions which when executed cause a computer system or device, or a computing system or device, to perform any of the methods (or (at least) a step of such methods) subject of this disclosure.

Related to computer or computing systems or to data-processing systems, this disclosure covers any such system comprising a means for carrying out or performing any herein disclosed method (or (at least) a step of such method).

### Computer/Computer System

A computer or computer system as mentioned herein may utilize one or more subsystems. A computer or computer system may be a single computer apparatus comprising the one or more subsystems (e.g. internal components), or may be multiple computers or multiple computer apparatuses each being a subsystem, and optionally, each comprising one or more own subsystems. Desktops, laptops, mainframe servers, tablets, mobile phones etc. all are computers or computer systems. The subsystems are usually interconnected and include a (central) processor (single-core processor, multi-core processor on a same integrated chip, or multiple processing units on a single circuit board or networked) capable of executing instructions, an input/output (I/O) controller, and a storage device (external, internal, peripheral, cloud, any medium readable by a computer or computer system). Input devices include keyboards, scanners, a computer mouse, camera, microphone, etc. In particular, the input device is a data collection or data generating device (which by itself may comprise a computer or computer system), such as a polynucleotide sequencing device (whether automated or not). Collected or generated data are fed to a computer or computer system designed to analyze the collected or generated data; this may be an ordinary computer system on which data analyzing software is installed (on a storage device) or which is capable of accessing data analyzing software (e.g. installed in or transmitted from a network) and whereby the processor of the computer system is instructed by the data analysis software on how to process the collected or generated data fed to the computer system, and how to display these via a display adapter to an output device. Output devices are further subsystems and comprise printers, monitors, computer readable medium. Input and output devices are usually connected to a computer or computer system via input/output ports to one another or via a network.

The specific combination of hardware and software allows implementation of e.g. analysis of data generated by a polynucleotide sequencing device, or of e.g. determination of the copy number of a SNP, or of e.g. determining the LOH or HRD status of a genomic DNA sample such as by a trained or machine learned data classification algorithm. Different software packages (proprietary or open source) can be run on a computer or computer system to achieve the desired degree of data analysis. Output of one computerized data analysis can be the input of a subsequent computerized data analysis step, hence creating an analysis pipeline. Software components can be written in different codes (e.g. Java, C, C++, Perl, Python) as long as the computer processor is able to execute the functions of the software component.

The methods of the invention may be computer-implemented methods, or methods that are assisted or supported (in part) by a computer or by a computer system. For instance, information reflecting the DNA sequencing analysis can be provided in user readable format by at least one/another processor. The same or a further processor may be calculating, as outlined herein, the LOH or HRD status of a test genomic DNA (such as relative to a control, standard or reference) from the information received. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array or such as a computer or machine readable medium, to access or store program instructions other data. Processors may further communicate via a network interface, which in turn may communicate via the one or more networks, such as the Internet or other public or private networks, such that a query or other request may be received from a client, or other device or service. Such computer-implemented methods (or such methods that are assisted or supported by a computer) may be provided as a kit or as part of a kit. The bioinformatics software required to perform (part of) the computer-implemented methods, i.e. a computer program product, may also be part of a kit, or may be provided as an individual product. A computer product may also consist of a computer or machine readable medium (in any form such as disks (hard, soft disks etc.), cards (memory cards etc.), tapes, sticks (memory, USB sticks etc.), microchips, DVDs, CDs, etc.) which is storing any of the instructions, computer program, or bioinformatics software enabling a computer system to perform at least one of the analysis of the herein described methods and/or to perform at least one calculation as described herein. Furthermore, the computer or computer system can be set up or configurated such that it is compliant with GDPR.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

In referring to genes or proteins herein, no distinction is made in the annotation. Thus, whereas for example the human BRCA1 gene would be referred to as the BRCA1 gene, the mRNA as BRCA1 mRNA, and the protein as BRCA1, such distinction is not, or not always, made hereinabove or hereinafter.

Sometimes, terms or words herein are between brackets. As will be clear from the context some of these bracketed terms or words are optional further clarification of the term or word. For instance “(test) genomic DNA sample” obviously refers to a genomic DNA sample, and the genomic DNA sample can thus be optionally clarified by referring to a test genomic DNA sample. Another example is “(further) inhibiting or impairing DNA repair”, basically meaning inhibiting or impairing DNA repair, and optionally referring to further inhibiting or impairing DNA repair.

It is to be understood that although particular embodiments, specific configurations as well as materials and/or molecules, have been discussed herein for methods etc. according to the present invention, various changes or modifications in form and detail may be made without departing from the scope and spirit of this invention. The following examples are provided to better illustrate particular embodiments, and they should not be considered limiting the application. The application is limited only by the claims.

The content of the documents cited herein are incorporated by reference.

### EXAMPLES

### Example 1. Materials and Methods

Targeted sequencing. Remaining DNA samples extracted from formalin-Fixed Paraffin-Embedded (FFPE) tissues were obtained from the PAOLA1 trial (Ray-Coquard et al. 2019, N Engl J Med 381: 2416-2428) through the Arcagy-Gineco (Paris, France) driven ENGOT HRD European initiative (Pujade-Lauraine et al. 2021, Int J Gynecol Cancer 31(Suppl 3):A208); samples from a subgroup (n=192) of the total PAOLA-1 trial population (n=806) were used herein. Specific genomic regions enriched using a capture-based probe design were sequenced (100 ng DNA input). This capture-based probe design included probes targeting a genome wide panel of single nucleotide polymorphisms (SNPs), sequenced at median target coverage of ˜150×. In total 96,861 SNPs were selected based on population frequency with a uniform distribution across the genome. Centromere regions were excluded. The SNPs were covered by 92,723 specifically designed probes. The average size of oligonucleotide probes was 120 bp. Capture libraries were sequenced on an Illumina Novaseq (paired-end, 2×150 bp reads).

### Bioinformatics Pipeline.

Mapping. Paired-end reads were processed with Burrows-Wheeler Aligner (BWA, version 0.4.1) using the hg19 human reference genome. Illumina adapter sequences were trimmed from the reads using TrimGalore (version 0.4.1). Duplicated reads were filtered and realigned locally around indels with the genomic analysis toolkit GATK (version 3.6—Broad Institute). Sequencing metrics were collected with Picard (version 2.5.0).

Copy number. Allelecount (version 4.2.1) was applied to determine the count coverage of each allele in the HRD genome wide panel regions. Minimum base quality and minimum mapping quality were set to 15. Copy numbers of target and antitarget regions were inferred using CNVkit (version 0.9.9). Next, the results from Allelecount and CNVkit were integrated as input for the allele-specific copy number analysis of tumors (ASCAT) tool (version 2.5.2; Van Loo et al. 2010, Proc Natl Acad Sci USA 107:16910-16915).

Loss-of-heterozygosity features. The ASCAT output was used to derive loss-of-heterozygosity (LOH) features based on the allele-specific copy number values. For each SNP, ASCAT reports the copy number value for both the major and minor allele. LOH was defined as the copy number of the minor allele being 0. Next, the genome was divided into bins of 1000 kb in size and the mean LOH across all SNPs within each bin was calculated for each sample, resulting in 2877 bin-specific LOH (or LOH/bin) features per sample. In addition to the bin features, also 23 chromosome specific features were created by calculating the mean LOH for each chromosome (LOH/chromosome features). Finally, a genome-wide LOH (LOH/genome feature) was calculated from the mean LOH across all SNPs per sample. Using this procedure, we obtained 2901 LOH features in total per sample, with each feature representing a local genomic region with averaged LOH values across multiple SNPs (LOH/bin) or features covering an entire chromosome (LOH/chromosome) or the entire genome (LOH/genome). All LOH calculations were performed in R (version 3.6.3).

Statistical analysis. Based on the 2877 bin-specific LOH features or the 2901 LOH features/sample (LOH/bin features+LOH/chromosome features+LOH/genome feature), an exemplary nearest shrunken centroid analysis was performed using the PAM (Partitioning Around Medoids) package in R (version 3.6.3) to check the power of this approach in distinguishing Myriad myChoice positive from Myriad myChoice negative samples. Using leave-one-out cross-validation, the best LOH features within each training set were selected and next a PAM score for each test sample was determined from the Myriad-positive status class probabilities. Whereas the Myriad myChoice HRD status was available for the samples, the actual Myriad myChoice HRD scores were not available and were approximated using the scarHRD algorithm (version 0.1.1; an R package which determines the levels of homologous recombination deficiency (telomeric allelic imbalance, loss of heterozygosity, number of large-scale transitions)). Receiver operation characteristic (ROC) for the PAM and HRD scores with respect to Myriad myChoice status were constructed using the pROC package (version 1.18.0). As an alternative to PAM, the Random Forest learning algorithm was used.

### Example 2. HRD Classification Based on Fractional Loss-of-Heterozygosity (FLOH)

Wang et al. 2012 (Clin Cancer Res 18: 5806-5815) proposed fractional LOH (FLOH) as feature to predict response of breast cancer to chemotherapy. FLOH was defined as the proportion of inferred LOH calls within the total number of SNPs. These authors, however, did not compare the FLOH feature to the Myriad MyChoice HRD classification. The ASCAT tool provides a feature for determining FLOH.

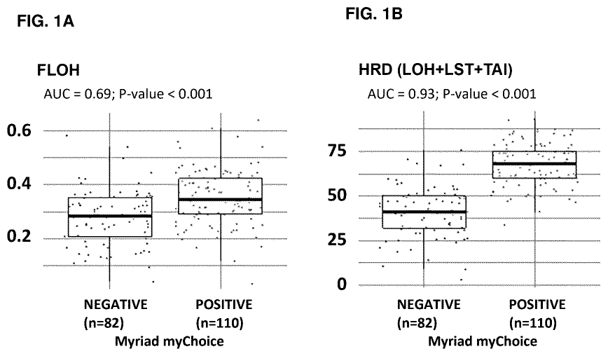

The performance of FLOH (via ASCAT tool feature) in classifying a subset of 192 samples of the PAOLA1 trial (HRD-positive: n=110; HRD-negative: n=82) was compared with the LOH+LST+TAI HRD score as approximated by the scarHRD algorithm. FIG. 1 indicates that, in contrast to the scarHRD score (FIG. 1B), the FLOH feature calculated using ASCAT (FIG. 1A) is not suited for distinguishing HRD-positive and HRD-negative samples.

These results appear to support the previously reported underperformance of the % LOH feature in distinguishing HRD-positive and HRD-negative samples (e.g. Stover et al. 2020, Gynaecologic Oncology 159:887-898).

### Example 3. HRD Classification Based on Number of LOH Regions of Intermediate Size

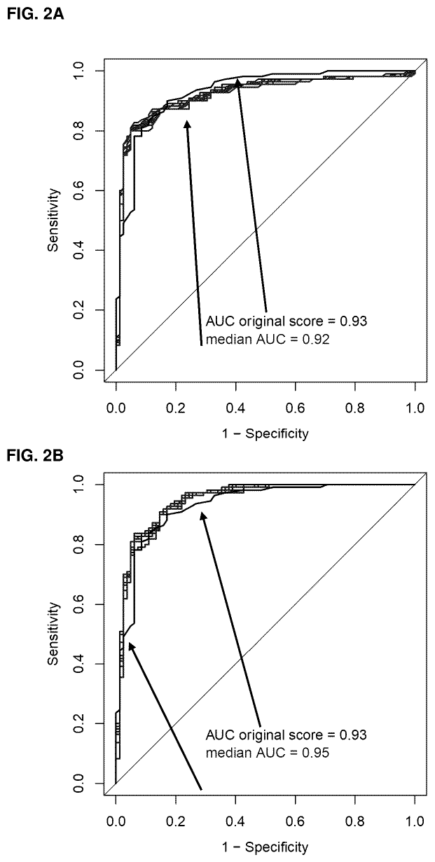

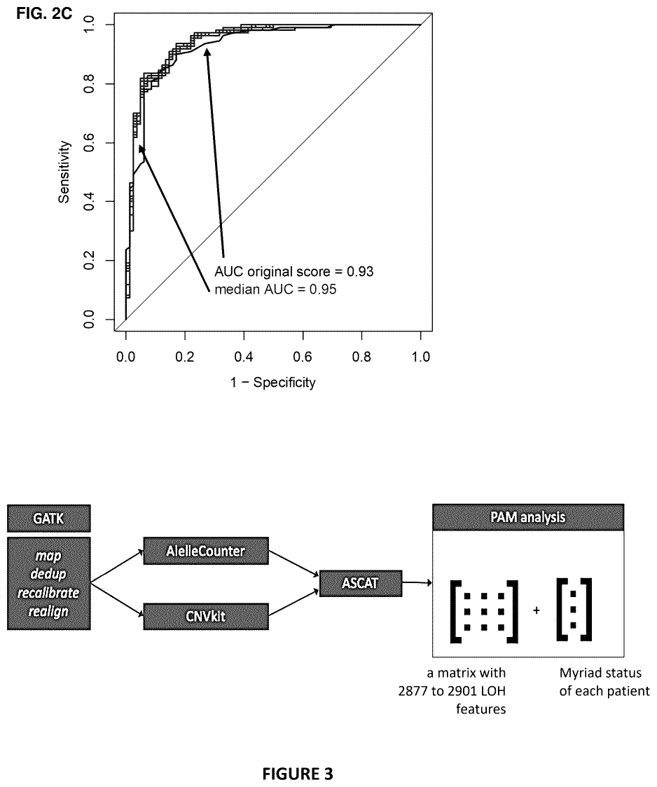

Abkevich et al. 2012 (Br J Cancer 107:1776-1782) defined a homologous recombination deficiency (HRD) score as the number of loss of heterozygosity regions of intermediate size regions observed in a tumour sample. This score was determined using 10-fold cross-validation for the same subset of 192 samples of the PAOLA1 trial as used in Example 2, and compared with the LOH+LST HRD score and LOH+LST+TAI HRD score as approximated by the scarHRD algorithm. As indicated in FIG. 2A, the AUC value of the LOH-only score (AUC=0.92) is close to the AUC value of the LOH+LST-score (AUC=0.95; FIG. 2B) and to the AUC value of the LOH+LST+TAI-score (AUC=0.95; FIG. 2C). The AUC value for the LOH+LST+TAI HRD score as approximated by the scarHRD algorithm=0.93 (“AUC original score”).

### Example 4. HRD Classification Based on LOH/Bin, LOH/Chromosome, and LOH/Genome Features

In searching for an alternative method for determining the HRD status of genomic DNA of a tumor or cancer cell relying on LOH features only, but performing as well as the combined LOH+LST+TAI features, it was found that the mean LOH/bin information, the mean LOH/chromosome and the mean LOH/genome information could be used efficiently in determining the HRD status of genomic tumor or cancer DNA. Moreover, for the mean LOH/bin information, the bin size could be lowered to under 1.5 Mb and data classification of the mean LOH/bin information, the mean LOH/chromosome and the mean LOH/genome information could be used efficiently in determining the HRD status of genomic tumor or cancer DNA.

Furthermore, these alternative methods relying on mean LOH/bin information, the mean LOH/chromosome and the mean LOH/genome information were found to predict as reliably as based on the Myriad myChoice status (itself based on LOH+LST+TAI) the response of high-grade serous ovarian cancer patients who received PARP inhibitor treatment (olaparib).

A cohort of 192 FFPE high-grade serous ovarian cancer samples from the PAOLA1 trial (identical to the samples used in Examples 2 and 3), composed of 132 patients administered with olaparib and 60 patients who received a placebo was examined. The Myriad myChoice status was determined previously for each sample as part of the PAOLA-1 trial. The Myriad myChoice HRD score was in contrast not available but approximated herein by the scarHRD algorithm (LOH (based on number of regions of LOH of intermediate size)+telomeric allelic imbalance (TAI)+large-scale transitions (LST)).

We herein used PAM as exemplary classification algorithm on the cohort of 192 samples to correlate LOH features with Myriad myChoice status. FIG. 3 shows the exemplary pipeline that was applied on all samples. The PAM score constitutes the class probabilities for Myriad-positive status and is extracted using leave-one-out cross-validation from the PAM analysis. A combination of LOH-features was fed to the classification algorithm: mean LOH/bin, mean LOH/chromosome, and mean genome-wide LOH (see above).

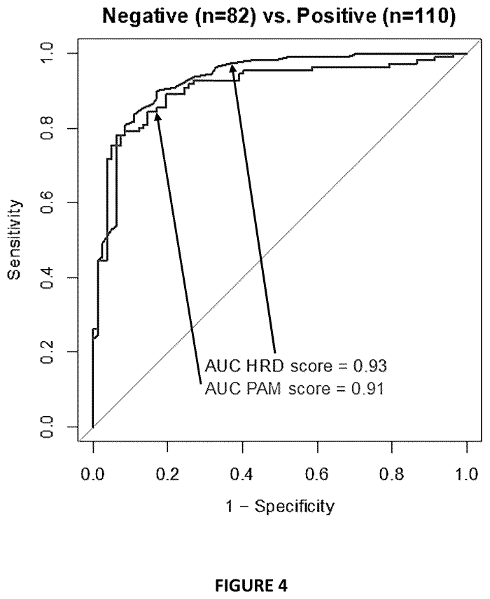

FIG. 4 demonstrates the performance of predicting HRD status using the estimated scarHRD score (“HRD score”: LOH (based on number of regions of LOH of intermediate size)+LST+TAI) or using the herein developed alternative method (“PAM score”: mean LOH/bin features+mean LOH/chromosome features+ and mean genome-wide LOH feature) with receiver-operator characteristic (ROC) curves. We found that the current alternative method based on LOH-only features (mean LOH/bin, mean LOH/chromosome, and mean genome-wide LOH) reached an AUC=0.91, which closely resembles the scarHRD score (LOH (based on number of regions of LOH of intermediate size)+LST+TAI); AUC=0.93) in predicting Myriad myChoice positive or negative status.

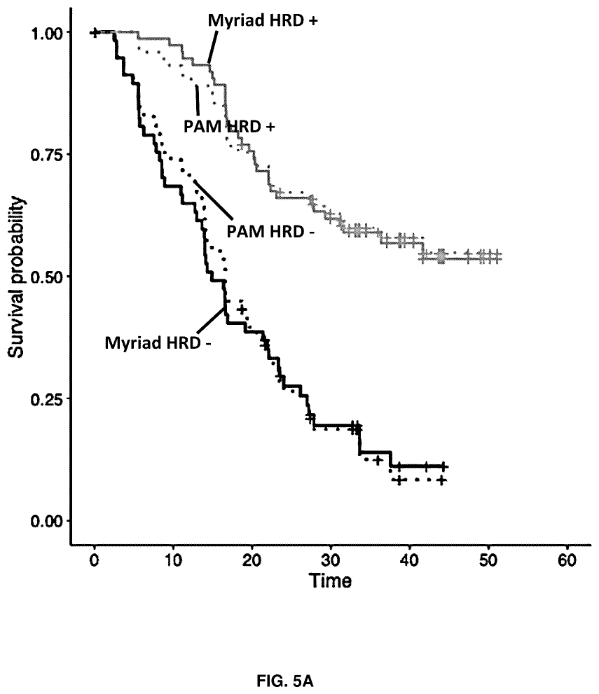

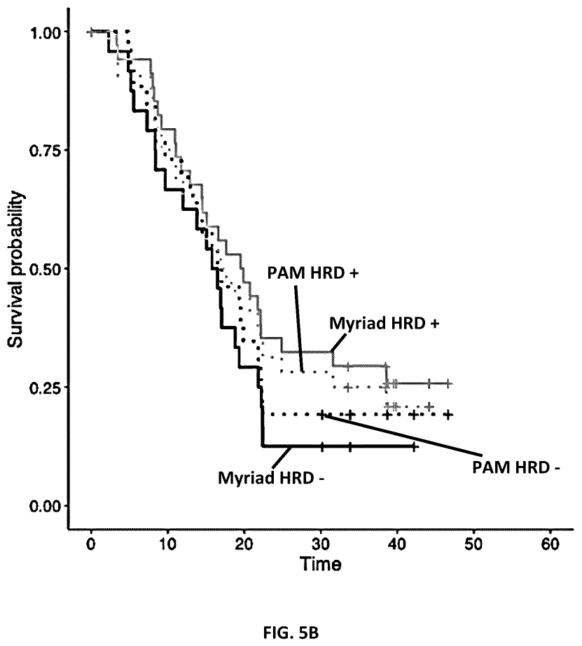

Encouraged by this unexpected positive result, we next assessed the survival curves of samples stratified by scarHRD or the current alternative method. Since the alternative data classifier score is a probability from 0 (HRD negative) to 1 (HRD positive), we herein simply used an exemplary threshold of 0.5 to stratify HRD positive and HRD negative samples. FIG. 5A shows the results for the patients treated with olaparib, with HRD positive patients (according to Myriad myChoice test) displaying a longer survival compared to HRD negative patients (according to Myriad myChoice test). The survival curves of patients stratified by the current alternative LOH-only based data HRD classifier score (mean LOH/bin, mean LOH/chromosome, and mean genome-wide LOH features) are as good as identical. FIG. 5B shows the results from patients in a control group administered with a placebo. All patients display shorter survival times compared to the olaparib treated patients and again a resemblance between the current alternative LOH-only based data classifier HRD score (mean LOH/bin, mean LOH/chromosome, and mean genome-wide LOH features) and scarHRD score (LOH (based on number of regions of LOH of intermediate size)+LST+TAI features) can be observed.

In conclusion, these results demonstrate that the current alternative LOH-only features and analysis as outlined herein can be effective predictors of HRD status in high-grade serous ovarian cancer patients who received platinum therapy, and this in a way fully comparable to the gold standard (LOH+LST+TAI) HRD score.

### Example 5. HRD Classification Based on LOH/Bin Features

In searching for a further alternative method for determining the HRD status of genomic DNA of a tumor or cancer cell relying on LOH features only, but performing as well as the combined LOH+LST+TAI features, it was found that, compared to Example 4, the mean LOH/bin information is sufficient, and that the mean LOH/chromosome and the mean LOH/genome information could be omitted, for efficiently in determining the HRD status of genomic tumor or cancer DNA.

Furthermore, these alternative methods relying on mean LOH/bin information were found to predict as reliably as based on the Myriad myChoice status (itself based on LOH+LST+TAI) the response of high-grade serous ovarian cancer patients who received PARP inhibitor treatment (olaparib).

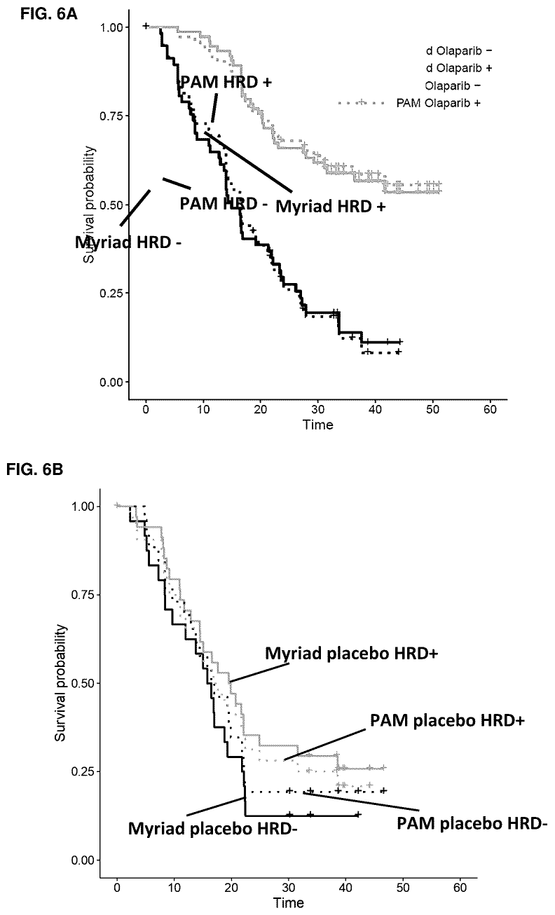

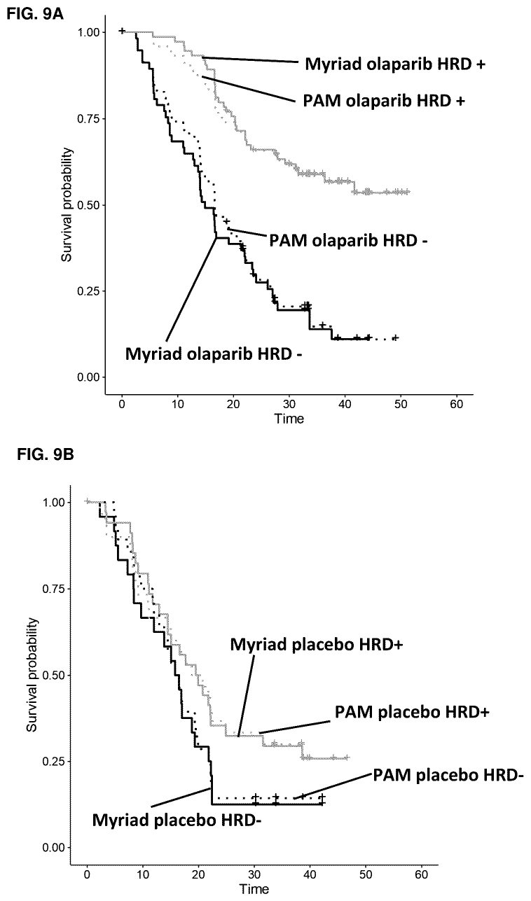

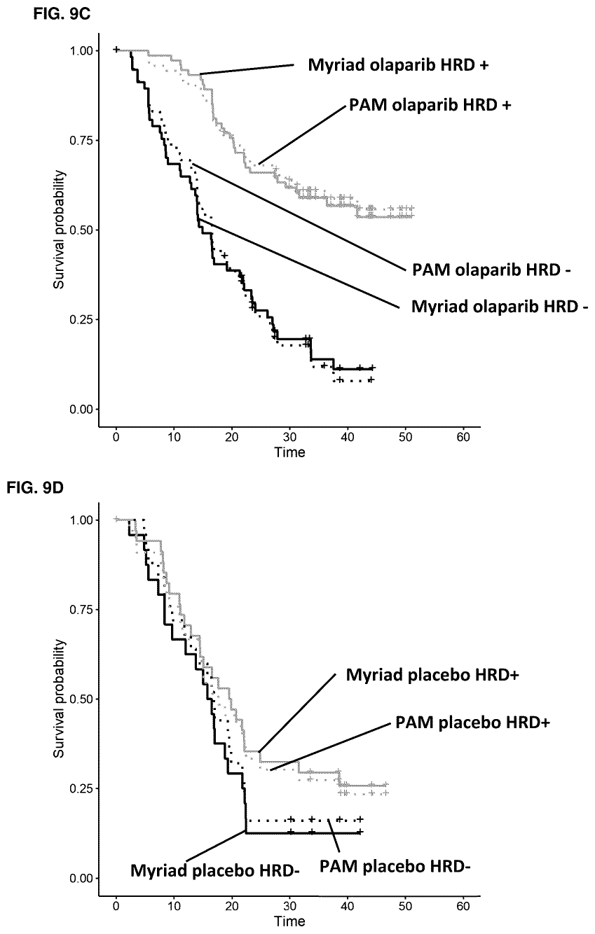

This was in particular demonstrated in FIG. 6 assessing the survival curves of samples stratified by scarHRD or the alternative method of Example 4 (results shown based on 10-fold cross-validation) but limited to using the mean LOH/bin features. FIG. 6A shows the results for the patients treated with olaparib, with HRD positive patients (according to Myriad myChoice test) displaying a longer survival compared to HRD negative patients (according to Myriad myChoice test). The survival curves of patients stratified byte alternative LOH-only based data HRD classifier score (mean LOH/bin features only; mean LOH/chromosome and mean genome-wide LOH features omitted) are as good as identical. FIG. 6B shows the results for the placebo group. The bin-size applied for the results depicted in FIG. 6 is 1 Mb (1000 kbp). The same alternative method of Example 4 but limited to mean LOH/bin features was re-iterated with an exemplary alternative bin size of 0.75 Mb (750 kbp) for which results are depicted in FIG. 9 A/B (A: olaparib-treated group; B: placebo group); and with an exemplary alternative bin size of 1.25 Mb (1250 kbp) for which results are depicted in FIG. 9 C/D (C: olaparib-treated group; D: placebo group).

Additionally, the hazard ratio's (HRs) for the HRD-positive groups were determined. Hazard is defined as the slope of the survival curve. The hazard ratio compares two treatments. In case of the hazard ratio being 2.0, then the rate of deaths in one treatment group is twice the rate in the other group. Herein, the hazard ratio was determined for the olaparib-treated HRD-positive subject group versus the control (placebo) HRD-positive subject group, a low hazard ratio implies a beneficial result of olaparib treatment. Results are summarized in Table 1.

TABLE 1
Survival hazard ratio's determined for HRD-positive subjects
based on PAM-analysis of mean LOH/bin for different bin sizes.

| Bin size | Hazard ratio HRD+ [95% confidence interval] |
|---|---|
| 750 kbp | 0.44 [0.26, 0.73] |
| 1000 kbp | 0.39 [0.24, 0.65] |
| 1250 kbp | 0.39 [0.23, 0.64] |

From FIGS. 6 and 9, and Table 1, it is obvious that analyses based on alternative bin sizes (all <1.5 Mb (1500 kbp)) lead to comparable results.

In conclusion, these results demonstrate that a further alternative LOH-only features and analysis as outlined herein can be effective predictors of HRD status in high-grade serous ovarian cancer patients who received PARP inhibitor therapy, and this in a way fully comparable to the gold standard.

### Example 6. HRD Classification Based on LOH/Bin Features Using Random Forest (RF) Classifier

Example 5 relied on the PAM algorithm as classifier of the mean LOH/bin features only. The same analysis (on the mean LOH/bin features only; same sample set) was performed using an alternative classifier, i.e. the Random Forest data classification algorithm (Breiman 2001, Machine Learning 45:5-32; Breiman and Cutler's Random Forests for Classification and Regression Version 4.7-1.1 Date 2022-01-24: https://cran.r-project.org/web/packages/randomForest/randomForest.pdf).

As an illustration of this alternative machine learning approach, the random forest classifier was trained on the Myriad myChoice status. The random forest classifier consisted of 5000 trees. At each node, 45 LOH bins were randomly selected as predictors and the minimum node size was set to 10. These parameters are exemplary and can be varied. LOH status of a bin was determined based on the copy number of the minor allele of the bin midpoint nucleotide position wherein this copy number was derived from the segment of the genomic DNA with constant copy number comprising the bin midpoint nucleotide position.

Within the training data set, 10-fold cross-validation was used for classification metrics, and a model was subsequently fitted to the full training set to obtain classification metrics on the validation set. ROC curves were constructed similar to the PAM method described earlier, and yielded an AUC value of 0.93 (i.e. identical to the AUC value obtained with the scarHRD score, see Example 4).

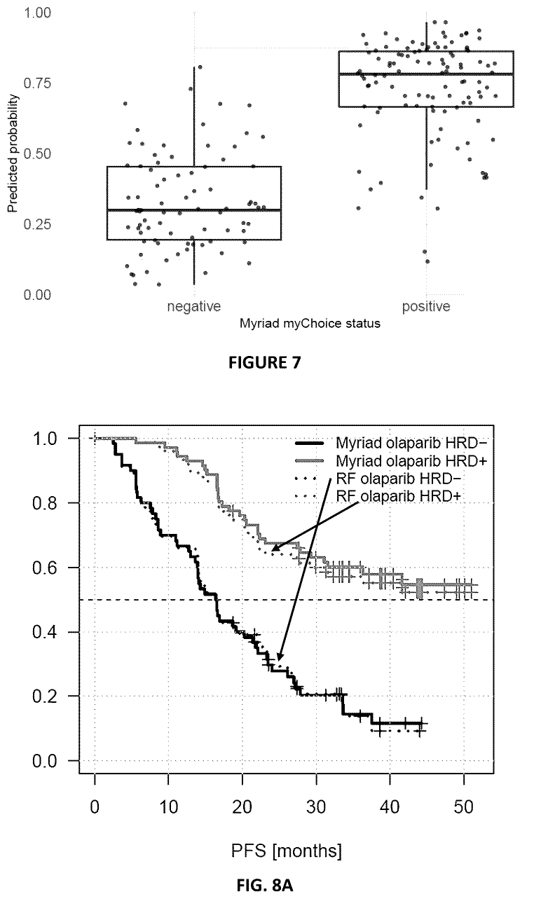

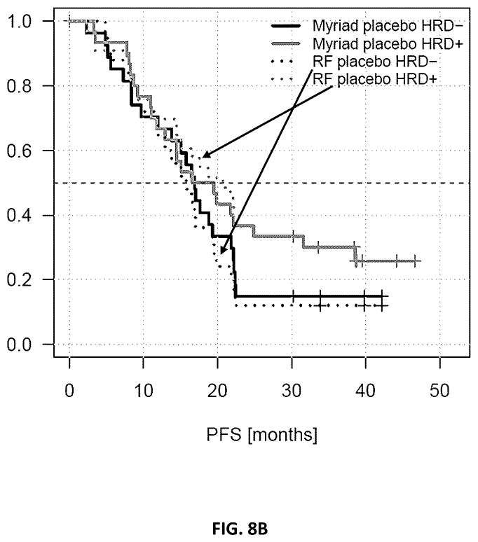

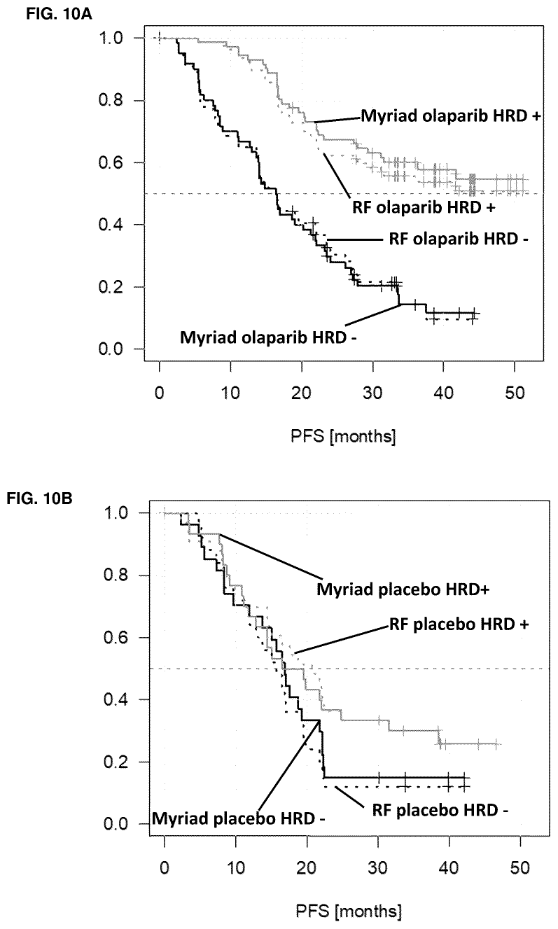

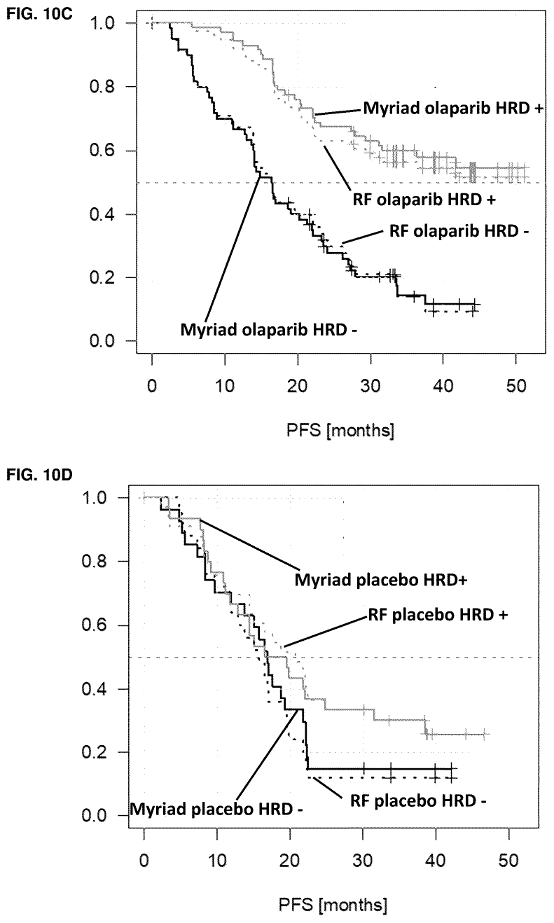

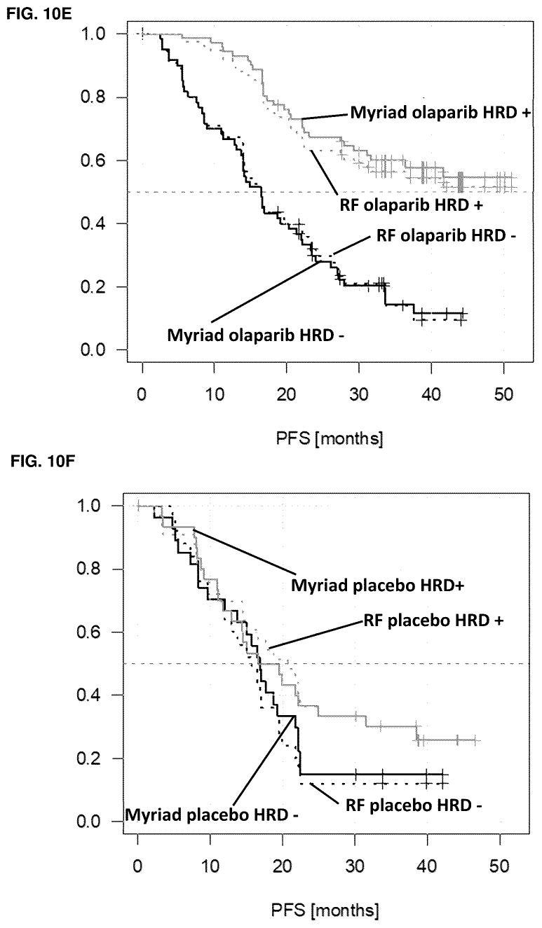

FIG. 7 indicates that the RF classifier can distinguish between Myriad myChoice HRD positive and negative samples (as well as the scarHRD algorithm, see FIG. 1B). Furthermore, survival probabilities of olaparib- or placebo-treated ovarian cancer patients based on the RF classification of the LOH/bin features are near identical to the survival probabilities based on the Myriad myChoice HRD status, as illustrated in FIG. 8 (A: olaparib-treated group; B: placebo group; results shown for 10-fold cross-validation). The bin-size applied for the results depicted in FIG. 8 is 1 Mb (1000 kbp). The same method RF classifier method was re-iterated with an exemplary alternative bin size of 0.5 Mb (500 kbp) for which results are depicted in FIG. 10 A/B (A: olaparib-treated group; B: placebo group); with an exemplary alternative bin size of 0.75 Mb (750 kbp) for which results are depicted in FIG. 10 C/D (C: olaparib-treated group; D: placebo group); and with an exemplary alternative bin size of 1.25 Mb (1250 kbp) for which results are depicted in FIG. 10 E/F (E: olaparib-treated group; F: placebo group).

Additionally, the hazard ratio's (HRs) for the HRD-positive groups were determined (cfr. Example 5), results are summarized in Table 2.

TABLE 2
Survival hazard ratio's determined for HRD-positive subjects
based on RF-analysis of mean LOH/bin for different bin sizes.

| Bin size | Hazard ratio HRD+ [95% confidence interval] |
|---|---|
| 500 kbp | 0.46 [0.27, 0.77] |
| 750 kbp | 0.45 [0.27, 0.76] |
| 1000 kbp | 0.44 [0.26, 0.74] |
| 1250 kbp | 0.45 [0.27, 0.76] |

From FIGS. 8 and 10, and Table 2, it is obvious that analyses based on alternative bin sizes (all <1.5 Mb (1500 kbp)) lead to comparable results.

1. A method for measuring the loss-of-heterozygosity (LOH) status of a test genomic DNA sample, the method comprising:
dividing the sequence of the test genomic DNA sample into a plurality of bins of equal size, the size being equal to or smaller than 1.5 Mb;
measuring the LOH status of each bin, therewith providing a set of LOH/bin features.

2. The method according to claim 1, wherein measuring the LOH status of a bin comprises measuring the copy number of one or more single nucleotide polymorphisms (SNPs) of interest, and is the mean of LOH positive SNPs relative to all SNPs of interest within the bin, wherein a SNP is LOH positive when the copy number of the minor allele of the SNP is zero.

3. The method according to claim 2, wherein measuring the copy number of a SNP of interest comprises measuring the copy number of a segment of the genomic DNA comprising the SNP of interest.

4. The method according to claim 1, wherein measuring the LOH status of a bin comprises measuring the copy number of a nucleotide position in the bin, and wherein the LOH status is the copy number of a segment of the genomic DNA having a constant copy number comprising the nucleotide position in the bin, wherein a bin is LOH positive when the copy number of the minor allele of the nucleotide position in the bin is zero.

5. The method according to claim 2, wherein the copy number measured via untargeted or targeted sequencing of the genomic DNA.

6. The method according to claim 2, er-3 wherein the one or more SNPs are captured by hybridization to a plurality of oligonucleotide probes.

7. The method according to claim 1, further comprising measuring the homologous recombination deficiency (HRD) status of the test genomic DNA sample, wherein a test genomic DNA sample that is LOH positive is HRD positive and wherein a test genomic DNA sample that is LOH negative is HRD negative.

8. (canceled)

9. (canceled)

10. The method according to claim 1, further comprising measuring the LOH status of each chromosome, therewith providing a set of LOH/chromosome features.

11. The method according to claim 1, further comprising measuring the LOH status of the test genomic DNA, therewith providing a LOH/genome feature.

12. A method of treating a subject having a tumor or cancer comprising
determining the LOH status of a genomic DNA sample obtained from the tumor or from a cancer cell of the cancer or tumor, according to the method of claim 16;
administering a DNA damaging agent and/or an agent inhibiting or impairing DNA repair to the subject if the determined LOH status of the genomic DNA sample is positive.

13. (canceled)

14. (canceled)

15. The method according to claim 1, further comprising:
computer-implemented classifying of the test genomic DNA based on the set of LOH/bin features wherein the classifying is performed with a data classification algorithm trained for classifying a set of reference genomic DNA samples including genomic DNA samples known of being LOH positive and/or homologous recombination repair deficient (HRD positive) and including genomic DNA samples known of being LOH negative and/or homologous recombination repair proficient (HRD negative) wherein the training is based on the LOH/bin features of the reference genomic samples;

16. The method of claim 15, further comprising determining the test genomic DNA sample to be LOH positive when the trained data classification algorithm classified the test genomic DNA sample as most closely resembling reference genomic DNA known of being LOH positive and/or HRD positive; or determining the test genomic DNA sample to be LOH negative or to have a LOH negative status when the trained data classification model classified the genomic DNA sample as most closely resembling reference genomic DNA known of being LOH negative and/or HRD negative.
